(19) United States
(12) Patent Application Publication
(10) Pub. No.: US 2010/0239682 A1
(43) Pub. Date: Sep. 23, 2010

(54) COLONIC DELIVERY OF ANTIMICROBIAL AGENTS

(76) Inventors: Antoine Andremont, Malakoff (FR); Jean De Gunzburg, London (GB)

Correspondence Address: INTELLECTUAL PROPERTY / TECHNOLOGY LAW, PO BOX 14329, RESEARCH TRIANGLE PARK, NC, 27709, US

(21) Appl. No.: 12/678,652

(22) PCT Filed: Sep. 16, 2008
(86) PCT No.: PCT/EP2008/062317

§ 371 Date: Jun. 4, 2010

Related U.S. Application Data
(60) Provisional application No. 60/994,066, filed on Sep. 17, 2007.

(51) Int. Cl. A61K 9/14; A61K 39/40; A61K 38/02; A61K 38/43; A61K 38/12; A61P 31/04; A61K 35/74

(52) U.S. Cl. 424/497; 424/164.1; 514/2.4; 424/94.1; 514/2.9; 424/93.4

(57) ABSTRACT

Antimicrobial compositions for oral delivery, and administration to the colon, distal ileum, or other portion of the gastrointestinal tract other than the stomach, of bacteriophage, phage proteins, antimicrobial peptides, or antimicrobial aptamers, are disclosed. In one embodiment, the active agent is capable of lysing the bacterial cell wall. In another embodiment, the active agent is capable of interacting with a receptor or enzyme in the bacteria. In some embodiments, the active agents selectively act on one or more harmful bacteria, such as Clostridium difficile, and either do not act, or act to a lesser extent, on helpful bacteria, such as bifidobacteria. When the agents are not delivered directly to the colon, they active agents ultimately enter the colon and affect the bacteria that are present in the colon. The compositions can include beads of pectin in the form of a cationic salt enclosing the active agent, or other types of drug delivery systems designed for targeted delivery to the desired portion of the gastrointestinal tract.

### FIELD OF THE INVENTION

The present invention is in the area of oral drug delivery systems to administer antimicrobial agents to the colon. More specifically, the present invention relates to the oral administration, and colonic delivery, of bacteriophages, antimicrobial proteins or peptides produced by the bacteriophages, other antimicrobial peptides, aptamers, or antibiotics which would are useful for treating infections or unwanted colonization in the colon, and for the effective decontamination of colonic flora from potential pathogenic bacteria, but which would be harmful or inefficient for these purposes if administered systemically.

### BACKGROUND OF THE INVENTION

There are typically many types of bacteria in the colon, including good bacteria and bad (pathogenic) bacteria. There are a number of colonic bacterial infections that can result in significant mortality and/or morbidity. Examples of these include infections caused by Clostridium difficile and certain strains of E. coli. Treatment with conventional antibiotics often results in bacterial resistance, which is not desirable.

In a number of instances the colon is colonized by resistant and potentially pathogenic bacteria, such as Enterobacteria, Enterococci, and Clostridium. Local antibiotics administered orally have been used to eliminate these bacteria. For example, vancomycin and metronidazole have been orally administered to eliminate Clostridium difficile. One of the drawbacks of such treatments is that the antibiotics are not highly specific of the target organisms, so their use affects other components of the intestinal flora. This can be deleterious, as it can put a selective pressure on commensal bacteria, and thus promote bacterial resistance.

The following have been observed in patients treated with conventional antibiotics:

1. Flora imbalance, which is the main cause of banal diarrhea occurring following antibiotic treatments (Bartlett J. G. (2002) Clinical practice. Antibiotic associated diarrhea, New England Journal of Medicine, 346, 334). Even though this diarrhea is generally not serious and ceases rapidly, either spontaneously, or upon completion of the antibiotic treatment, it is adversely perceived by patients and adds to the discomfort of the original illness for which the antibiotic was prescribed;

2. interference with the resistance to colonization (or “barrier effect”) of the indigenous intestinal flora by exogenic bacteria with possible risk of infection, such as alimentary salmonella intoxication (Holmberg S. D. et al. (1984) Drug resistant Salmonella from animals fed antimicrobials, New England Journal of Medicine, 311, 617);

3. Selection of microorganisms resistant to the antibiotic. These microorganisms can be of various types:

a) first they can be pathogenic bacteria such as for example, Clostridium difficile, a species capable of secreting toxins causing a form of colitis known as pseudomembranous colitis (Bartlett J. G. (1997) Clostridium difficile infection: pathophysiology and diagnosis, Seminar in Gastrointestinal Disease, 8, 12);

b) they can also be microorganisms that are relatively weakly pathogenic, but whose multiplication can lead to an associated infection (vaginal candidosis or Escherichia coli resistant cystitis).

c) they can finally be non-pathogenic commensal drug-resistant bacteria whose multiplication and fecal elimination will increase dissemination of antibiotic resistance in the environment. It is well documented that antibiotic resistance genes are carried by mobile or transposable genetic elements that may contain up to 5 or 6 antibiotic resistance genes, and are readily transmitted to other bacteria, even across species. Consequently, these resistant commensal bacteria may constitute an important source leading to drug resistance for pathogenic species. This risk is currently considered seminal in terms of the disquieting character of the evolution towards drug multiresistance by numerous species pathogenic for humans (Donskey C J. Antibiotic regimens and intestinal colonization with antibiotic-resistant gram-negative bacilli. Clin Infect Dis. 2006 Sep. 1; 43 Suppl 2:S62-9).

It would be advantageous to provide antimicrobial agents to the colon in two different clinical conditions: first to treat established colonic infections, and second, to eliminate asymptomatic colonization by unwanted microbes (resistant and/or potentially pathogenic ones) as a measure aimed at preventing the spread of the unwanted microbes in the environment and at preventing the occurrence of subsequent infections in the colonized hosts. In order to do so one would need to have, in the colon, adequate concentrations of active agents that, besides the desire effect, do not promote bacterial resistance, and/or are selective for harmful (pathogenic) or unwanted microbes or bacteria over helpful bacteria, which ideally should remain largely unaffected. Several examples of the advantageous providing of antimicrobials directly in the colon by a specific targeting system are listed below:

Concerning colonic infections, providing antimicrobials agents to the colon in such a manner would be of particular interest in infections where the primum movens of the disease is the multiplication of the pathogenic microbe within the lumen of the intestinal tract, with little invasion of the mucosa, the pathogenic alterations of the mucosa being resulting from the action of compounds (such as toxins) released by the infecting bacteria. A typical type of such infections would be those caused by Clostridium dificile such as post-antibiotic diarrhea, colitis, or pseudo-membranous colitis, which are commonly grouped under the name of Clostridium difficile associated diseases, or CDAD (see Bartlett J G, Curr Infect Dis Rep. 2002 December; 4(6):477-483. Clostridium difficile-associated Enteric Disease). Indeed, the current recommendations are to use oral antibiotics such as vancomycin or metronidazole to treat patients suffering from a severe enough case of CDAD (DuPont H L, Garey K, Caeiro J P, Jiang Z D. New advances in Clostridium difficile infection: changing epidemiology, diagnosis, treatment and control. Curr Opin Infect Dis. 2008 October; 21(5):500-7). Although efficient in the majority of cases, this treatment suffers from several flaws. First, the antibiotics administered are far from being selective for C. difficile. Thus, they exert a definite selective pressure on the rest of the intestinal flora, and may be at the origin of the selection of resistant bacteria. Second, the treatment is not always effective (the cure rate is estimated to be as low as 70% in severe cases of CDAD). A treatment which would be more effective and more selective is needed.

Concerning asymptomatic colonization by unwanted microbes and their elimination, a medical practice referred to as “selective decontamination” as been described and used with this goal for over 30 years. It aims at eliminating commensal and/or potentially pathogenic microorganisms (such as for example enterobacteria, pseudomonas, enterococci) from the colon of patients at risk (such as Intensive care, or haemato-oncology patients, for example) before they develop an actual infection. However, selective decontamination has never gained general acceptance in spite of its most probable favorable effect to prevent the occurrence of Gram-negative infections in patients at risks (Anaesth Intensive Care. 2008 May; 36(3):324-38. Impact of selective decontamination of the digestive tract on carriage and infection due to Gram-negative and Gram-positive bacteria: a systematic review of randomized controlled trials. Silvestri L, van Saene H K, Casarin A, Berlot G, Gullo A.). This lack of confidence of the medical community in the effectiveness of selective decontamination is due to several factors: first, the decontamination is without effect on the occurrence of gram-positive infections, because it currently relies on anti-Gram negative antimicrobials. This reduces the overall interest of the practice. Second, the antimicrobials used for selective decontamination belong to the same antimicrobial families which are also used for treating infected patients (for instance, aminoglycosides or fluoroquinolones or colimycin). This is associated with the fear that large uses of these agents for selective decontamination will ultimately select resistant bacteria, which will result in infections that are untreatable by most of the currently-available antibiotics.

Another possible use of selective decontamination can also be found in farm animals. Indeed, bovines and ovines are often asymptomatically colonized by specific types of Escherichia coli strains, namely Shiga-toxin Escherichia coli (or STEC), also called Verotoxin Escherichia coli (or VETEC) (Goldwater P N. Treatment and prevention of enterohemorrhagic Escherichia coli infection and hemolytic uremic syndrome. Expert Rev Anti Infect Ther. 2007 August; 5(4):653-63). These strains can contaminate foodstuff, including meat during slaughtering, milk during the milking process, and fruits and vegetables to be eaten raw after the use of contaminated water for irrigation or animal waste for manure.

Cases have also been observed after recreational activities in water contaminated by animal waste. Ingestion of only a few number of STEC (10-100 cells) can cause bloody diarrhea in humans, particularly in young children. In addition ˜5% of the subjects that develop such diarrhea will suffer in the following weeks of a much more severe disease called hemolytic and uremic syndrome (or SHU). SHU is a severe disease often requiring hospitalization and extra-renal epuration. The death rate is around 5%. There is no current means of proven efficacy for decreasing STEC colonization in the livestock (Fairbrother J M, Nadeau E Escherichia coli: on-farm contamination of animals, Rev Sci Tech. 2006 August; 25(2):555-69). However, targeting effective antibacterials to the colon of colonized animals may achieve this goal.

The above mentioned rationale to use selective decontamination could be applied also towards colonization by vancomycin resistant enterococci (VRE), the dissemination of which is also currently a major threat, because these bacteria, albeit little pathogenic by themselves, carry genes that confer high levels of resistance to all glycopeptides, a last line antibiotic family for the treatment of Gram positive infections such as those caused by methicillin resistant S. aureus (Tacconelli E, Cataldo M A. Vancomycin-resistant enterococci (VRE): transmission and control. Int J Antimicrob Agents. 2008 February; 31(2):99-106.). Indeed, these resistance genes can readily transfer from enterococci to staphylococci and this risk has justified the implementation of very strict control measures in hospitals where VRE are prevalent. However, these measures do not include intestinal decontamination of the carriers, because no molecule has been shown to be effective to do so, so far. This may be why the controls measures, however heavy, currently have a limited proven efficacy. Selective decontamination by anti-VRE molecules specifically targeted to the colon may overcome these problems.

Selective decontamination has also been advocated to help control outbreaks of antibiotic resistant gram-negative infections in hospitals (Brun-Buisson C, Legrand P, Rauss A, Richard C, Montravers F, Besbes M, Meakins J L, Soussy C J, Lemaire F Intestinal decontamination for control of nosocomial multiresistant gram-negative bacilli. Study of an outbreak in an intensive care unit. Ann Intern Med. 1989 Jun. 1; 110(11):873-81.) This use was advocated mostly against outbreaks of nosocomial infections caused by enterobacteria (mostly Klebsiella) resistant to third generation cephalosporins by secretion of an extended spectrum beta-lactamase (ESBL) derived from the TEM or SHV beta-lactamase families. Such bacteria were mostly observed in the hospital setting and very rarely in the community. However, this type of ESBL and the associated outbreaks have currently mostly disappeared from the hospitals of many countries when the use of hand hygiene with alcoholic solutions and contact precautions have been implemented to a large scale in hospitals to prevent bacterial cross transmission between patients. However, a new type of ESBL, called CTX-M, which have a strikingly different epidemiological pattern of emergence and diffusion, are currently prevalent in hospitals (Pitout J D, Laupland K B. Extended-spectrum beta-lactamase-producing Enterobacteriaceae: an emerging public-health concern. Lancet Infect Dis. 2008 March; 8(3):159-66). CTX-M enterobacteria are not only resistant to third generation cephalosporins, just as ESBL ones, but they are widely present both in the community where they seems to emerge first and in the hospitals which appear to be invaded from the outside by virtue of community patients already colonized when they are admitted. In addition, CTX-M are most often carried by enterobacterial species (such as Escherichia coli) much better fitted to the intestinal tract ecosystem than those carrying previously known ESBL. Thus, they apparently have very little tendency to be spontaneously eliminated. Because of these combined facts, colonization by CTX-M enterobacteria is considered to be a major threat for the sake of antibacterial treatments in the coming years. A possible strategy would be to selectively decontaminate the colon of the colonized patients upon admission, after screening, using specific antibacterial agents targeted to the colon.

A renewed approached to selective decontamination for the five above-mentioned examples would be to use agents highly selective for the target organisms (alone or in combination to prevent selection and emergence of resistant mutants), ideally of completely different chemical composition than the antibiotics used for treating infected patients, in order to avoid cross-resistance. However, such agents are often fragile and cannot be administered orally without proper formulation, because they would be destroyed before reaching the colon due to the physico-chemical conditions prevailing in the upper part of the intestinal tract.

It would be advantageous to provide compositions and methods for delivering such agents to the colon in their active form after oral administration. The present invention provides such compositions and methods.

### SUMMARY OF THE INVENTION

Compositions and methods for treating bacterial infection or eliminating unwanted colonization in the colon are disclosed. The compositions comprise antimicrobial agents and/or compounds which bind to or otherwise destroy bacterial toxins. The active agents are administered orally, but are delivered to the colon. Methods for preparing the drug delivery systems, and for treating bacterial infections in the colon using such drug delivery systems, are also disclosed.

In one embodiment, the present invention is directed to drug delivery systems for oral administration, and delivery to the colon, of active agents such as bacteriophages, antimicrobial phage proteins, and antimicrobial proteins, peptides or aptamers not produced by bacteriophages. The active agents can also be, for example, antibodies, antibody fragments, or enzymes. The antibodies can be, for example, monoclonal, humanized (chimeric) or polyclonal antibodies, and can be prepared, for example, using conventional techniques.

In a second embodiment, the present invention is directed to drug delivery systems for oral administration, and delivery to the colon, of antibacterial agents which are toxic if administered systemically, but effective and non-toxic if administered specifically to the colon site, to treat an infection in the colon. For example, polypeptide antibiotics such as colistin, and some aminoglycosides, are toxic if administered systemically (i.e. by intravenous injection, but highly effective if delivered locally.

In one aspect of both embodiments, the active agents are highly active bactericidal substances which have a much narrower spectrum of activity than classical antibiotics, in that they are effective against, and selective for, harmful (pathogenic) bacteria over helpful (beneficial) bacteria. In this manner, the problems associated with selective pressure, caused by conventional antibiotics, are alleviated. Certain bacteriophage, phage proteins, antimicrobial peptides, and antimicrobial aptamers are suitable for accomplishing this goal. Those agents, such as proteins, peptides, and oligonucleotides, would likely be destroyed after oral administration in the upper part of the intestinal tract if they were not administered using the drug delivery systems described herein.

In a third embodiment, the present invention is directed to drug delivery systems for oral administration, and delivery to the colon, of antitoxins and toxin-binding compounds. Examples include antibodies which bind to bacterial toxins, and anionic polymers such as tolevamer.

In some aspects of these embodiments, combinations of these types of agents are used. For example, one can use a combination of antimicrobial agents to treat more than one type of bacteria, or to treat one type of bacteria in a way that minimizes its ability to develop resistance. One can also use a combination of an antimicrobial agent and a substance which binds to or otherwise inactivates a bacterial toxin, to both kill the bacteria and minimize the harmful effects of the bacterial toxin.

In one aspect of these embodiments, the agents are administered orally, but specifically released at the colon site. Pectin beads are one example of a drug delivery formulation that can release the active agents specifically at the colon site following oral administration, though other formulations for specific colonic delivery can also be used.

In another aspect of these embodiments, they are administered orally, and are released after the stomach, but in advance of the colon, for example, the distal ileum, or other location in the gastrointestinal tract other than the stomach, and arrive at the colon following normal gastrointestinal transit, where they can treat colonic bacterial infections or be selective decontamination agents. This aspect is preferred for drugs which are toxic if administered systemically.

The compositions can be used to provide antimicrobial agents to the colon to eliminate pathogenic microbe within the lumen of the intestinal tract, minimizing the pathogenic alterations of the mucosa resulting from the action of compounds (such as toxins) released by infecting bacteria such as Clostridium difficile. Thus, the compositions can be used to treat post-antibiotic diarrhea, colitis, or pseudo-membranous colitis, which are commonly grouped under the name of Clostridium difficile associated diseases, or CDAD.

The compositions can also be used to provide antimicrobial agents to the colon to provide “selective decontamination,” eliminating commensal and/or potentially pathogenic microorganisms (such as for example enterobacteria, pseudomonas, enterococci) from the colon of patients at risk (such as intensive care, or haemato-oncology patients, for example) before they develop an actual infection.

The compositions can further be used to provide selective decontamination of the colonic bacteria in farm animals, particularly, of specific types of Escherichia coli strains, namely Shiga-toxin Escherichia coli (or STEC), also called Verotoxin Escherichia coli (or VETEC). In use, the practice of this method can minimize contamination of the food and water supplies.

The compositions can also be used to provide selective decontamination against colonization by vancomycin resistant enterococci (VRE), providing selective decontamination using anti-VRE molecules specifically targeted to the colon.

The compositions can also be used to provide selective decontamination to help control outbreaks of antibiotic-resistant gram-negative infections, such as nosocomial infections, in hospitals. Representative infections include nosocomial infections caused by enterobacteria (mostly Klebsiella) resistant to third generation cephalosporins by secretion of an extended spectrum beta-lactamase (ESBL) derived from the TEM or SHV beta-lactamase families, as well as a new type of ESBL, called CTX-M, which have a strikingly different epidemiological pattern of emergence and diffusion. In one embodiment, patients admitted to a hospital, who have identified positive for one of these bacteria, are selectively decontaminated using specific antibacterial agents targeted to the colon.

Ideally, the antimicrobial active agent(s) delivered to the colon are specific for pathogenic bacteria, and do not have an effect on beneficial bacteria. In this manner, the agents do not cause selective pressure in the colon, and the beneficial bacteria are preserved. Accordingly, in one embodiment, the proteins, peptides, or aptamers are active against one or more species of harmful bacteria present in the colon, but are either not active, or are less active, against helpful bacteria present in the colon. It can be particularly useful to use more than one agent, so that the problem of bacterial resistance developing against one agent is minimized.

Although it is desirable to selectively treat pathogenic bacteria, rather than beneficial bacteria, in one embodiment, the active agents are non-selective, and eliminate both beneficial and pathogenic bacteria, and the beneficial bacteria can be replaced, for example, using probiotic formulations.

The drug delivery systems are orally administrable, and in some embodiments, deliver the active agents to the colon, and in other embodiments, they administer the agents to various positions in the gastro-intestinal tract other than, or in addition to, the colon.

In one embodiment, the drug delivery systems for providing colon-specific delivery comprise pectin beads crosslinked with zinc or any divalent, trivalent, or polycationic cation of interest, which beads can optionally be coated with a polycationic polymer, and/or coated with any suitable polymer for delivery to the desired part of the gastro-intestinal tract such as Eudragit®-type polymers.

In other embodiments, sustained delivery systems can be used, provided they at least get past the stomach without adversely effecting the active agent(s). For example, the active agents can be admixed with a polymer that degrades or dissolves over time, releasing the active agent. These types of systems are often coated with an enteric coating, to get past the stomach, and release agents throughout the gastrointestinal tract.

Colon-specific delivery can be obtained by formulating the active agent with specific polymers that degrade in the colon, such as pectin. The pectin is crosslinked with a cation such as a zinc cation. The formulation, typically in the form of ionically crosslinked pectin beads, can be further coated with a suitable polymer, such as polylysine, chitin, or polyethylene imine, and/or coated with a specific polymer, such as a Eudragit® polymer.

The delivery can be modulated to occur at various pre-selected sites of the intestinal tract by gelling/crosslinking a mixture of the encapsulated agent and pectin, with divalent metallic cations such as Ca²+ or Zn²+ and choosing the appropriate polymer that will protect the active agent from acidic conditions in the upper part of the gastro-intestinal tract and will dissolve at a given site in the lower part of the gastro-intestinal tract.

The drug delivery systems can be formulated in accordance with the teachings of U.S. patent application Ser. No. 10/524,318, and U.S. Patent Application No. 60/651,352, the contents of which are hereby incorporated by reference. These references disclose coating pectin beads with cationic polymers such as polyethylene imine (PEI), chitosan or other cationic polymers, to prevent the pectin beads from degrading in the upper gastro-intestinal tract.

The drug delivery systems can also be coated with Eudragit® polymers such as FS30D, L30D, NE30D, mixtures thereof or other desirable types of Eudragit® to achieve the desired release of the encapsulated agent at predefined levels of the gastro-intestinal tract (GIT).

When using pectin beads coated with Eudragit® polymers, the Eudragit® coating is dissolved, according to certain parameters such as pH or time, the beads are preferentially degraded by pectinolytic enzymes found in the lower part of the intestinal tract. Degradation of pectin then releases the agent encapsulated within the bead.

One aspect of the invention is to provide a stable formulation for the lower intestinal or colonic delivery of the encapsulated agents. The use of zinc cations to crosslink the pectin is particularly preferred if the antimicrobial protein or peptide is metallo-dependent, specifically, zinc dependent. Of course, if the proteins or peptides are dependent on other metal cations, such other metal cations (if they have a valence exceeding +1) can be used to crosslink the pectin.

The processes to obtain such beads can involve specific process conditions, such as time for gelification, washing, and drying that can be optimized to provide the highest quality beads, with optimized efficacy in vitro and in vivo. Therefore, another embodiment of the invention relates to processes for preparing zinc-crosslinked and Eudragit®-coated pectin beads encapsulating the active agents.

The agents, or combinations thereof, can be formulated in capsules, tablets or any acceptable pharmaceutical composition, and are ideally designed to specifically release the active agents in a programmed manner at a specific site of the intestinal tract, such as but not limited to the distal ileum or colon. The programmed delivery prevents the agents from being degraded in the stomach after oral absorption, and permits them to be released into the lower part of the small intestine, i.e. the ileum, and the colon. In one embodiment, the compositions allow the formulated active agents to recover their maximum antimicrobial capabilities when they reach the desired part of the intestinal tract.

The manner in which the antimicrobial agents, proteins, peptides, or aptamers are identified can vary. However, in one embodiment, a target is identified which is present in the one or more species of harmful bacteria, and which is not present in the helpful bacteria. The target can be used in peptide phage display, or SELEX, to identify peptides and/or aptamers suitable for use in inhibiting the growth, killing, or otherwise adversely affecting the harmful bacteria.

Combinatorial libraries of compounds, for example, phage display peptide libraries and oligonucleotide libraries can be screened. Phage peptides or oligonucleotides which bind to relevant targets can be identified, for example, using affinity binding studies, or using other screening techniques known to those of skill in the art. The effect of the compounds once bound to the targets can be determined, for example, by evaluating the growth of various bacterial populations exposed to the compounds.

The present invention will be better understood with reference to the following detailed description.

### DETAILED DESCRIPTION

The drug delivery systems, and methods of treatment described herein, can minimize or prevent the generation of “resistant” variants of pathogenic bacteria, and/or remove bacterial toxins. The compositions can include one specific active agent, or a combination of active agents. For example, combinations of active agents can help prevent the emergence of resistance, a single agent can be used as a selective decontamination agent, and combinations of antimicrobial compounds and compounds that remove bacterial toxins can minimize the bacterial infection, as well as minimize damage to the colon caused by the bacterial toxins.

The following description includes the best presently contemplated mode of carrying out the invention. This description is made for the purpose of illustrating the general principles of the inventions and should not be taken in a limiting sense.

The drug delivery systems described herein will be better understood with reference to the following detailed description, and the following definitions:

The terms “a”, “an” and “the” as used herein are defined to mean “one or more” and include the plural unless the context is inappropriate.

Unless defined otherwise, all technical and scientific terms used herein have the same meaning as commonly understood by one having ordinary skill in the art. Although other materials and methods similar or equivalent to those described herein can be used in the practice or testing of the present invention, as would be apparent to practitioners in the art, the preferred methods and materials are now described.

As used herein, the term “bacterium” refers to a single bacterial strain, and includes a single cell, and a plurality or population of cells of that strain unless clearly indicated to the contrary. In reference to bacteria or bacteriophage the term “strain” refers to bacteria or phage having a particular genetic content. The genetic content includes genomic content as well as recombinant vectors. Thus, for example, two otherwise identical bacterial cells would represent different strains if each contained a vector, e.g., a plasmid, with different phage ORF inserts.

The terms “bacteriophage” and “phage” are used interchangeably to refer to a virus which can infect a bacterial strain or a number of different bacterial strains.

The term “fragment” refers to a portion of a larger molecule or assembly. For proteins, the term “fragment” refers to a molecule which includes at least 5 contiguous amino acids from the reference polypeptide or protein, preferably at least 8, 10, 12, 15, 20, 30, 50 or more contiguous amino acids. In connection with oligo- or polynucleotides, the term “fragment” refers to a molecule which includes at least 15 contiguous nucleotides from a reference polynucleotide, preferably at least 24, 30, 36, 45, 60, 90, 150, or more contiguous nucleotides.

The term “bacteriophage ORF” or ““phage ORF” or similar term refers to a nucleotide sequence in or from a bacteriophage. In connection with a particular ORF, the terms refer an open reading frame which has at least 95% sequence identity, preferably at least 97% sequence identity, more preferably at least 98% sequence identity with an ORF from the particular phage identified herein (e.g., with an ORF as identified herein) or to a nucleic acid sequence which has the specified sequence identify percentage with such an ORF sequence.

The terms “inhibit”, “inhibition”, “inhibitory”, and “inhibitor” all refer to a function of reducing a biological activity or function. Such reduction in activity or function can, for example, be in connection with a cellular component, e.g., an enzyme, or in connection with a cellular process, e.g., synthesis of a particular protein, or in connection with an overall process of a cell, e.g., cell growth. In reference to bacterial cell growth, for example, an inhibitory effect (i.e., a bacteria-inhibiting effect) may be bacteriocidal (killing of bacterial cells) or bacteriostatic (i.e., stopping or at least slowing bacterial cell growth). The latter slows or prevents cell growth such that fewer cells of the strain are produced relative to uninhibited cells over a given period of time. From a molecular standpoint, such inhibition may equate with a reduction in the level of, or elimination of, the transcription and/or translation of a specific bacterial target(s), or reduction or elimination of activity of a particular target biomolecule.

As used herein, “peptide” is defined as including less than or equal to 100 amino acids and “protein” is defined as including 100 or more amino acids.

A “target” refers to a biomolecule in a bacteria that can be acted on by an active agent as described herein, thereby modulating, preferably inhibiting, growth or viability of a bacterial cell. In most cases such a target will be a nucleic acid sequence or molecule, or a polypeptide or protein. However, other types of biomolecules can also be targets, e.g., membrane lipids and cell wall structural components.

### I. Pectin Beads

In one embodiment, the drug delivery systems are designed to be orally administered, but deliver the active agents specifically to the colon, and substantially nowhere else in the gastrointestinal tract. Pectin beads are one example of a formulation that can deliver active agents specifically to the colon.

The pectin beads are formed from pectin, zinc or other metal ions, and can be coated with a cationic polymer and/or further coated with Eudragit® polymers. The pecting beads also encapsulate one or more active agents.

The stability and protection of the pectin beads in gastric medium and intestinal medium can be ensured by the reticulation with polycationic polymers and/or Eudragit® polymer or other polymer coatings. In contrast, uncoated beads of pectin are typically not stable in such an environment and may not adequately protect their contents against degradation and/or inactivation. The reticulation and/or polymer coatings ensure that they resist long enough so that their contents are able to reach the colon intact.

Pectin

Pectin is a polysaccharide isolated from the cellular walls of superior plants, used widely in the agricultural food industry (as a coagulant or thickener for jams, ice creams and the like) and pharmaceutics. It is polymolecular and polydisperse. Its drug delivery system varies depending on the source, extraction conditions and environmental factors.

Pectins are principally composed of linear chains of beta-1,4-(D)-galacturonic acid, at times interspersed by units of rhamnose. The carboxylic groups of galacturonic acid can be partially esterified to yield methylated pectins. Two types of pectins are distinguished according to their degree of methylation (DM: number of methoxy groups per 100 units of galacturonic acid):
highly methylated pectin (HM: high methoxy) where the degree of methylation varies between 50 and 80%. It is slightly soluble in water and forms gels in acidic medium (pH<3.6) or in the presence of sugars;
weakly methylated pectin (LM: low methoxy), with a degree of methylation varying from 25 to 50%. More soluble in water than HM pectin, it gives gels in the presence of divalent cations such as Ca²⁺ ions. Indeed, Ca²⁺ ions form “bridges” between the free carboxylated groups of galacturonic acid moities. The network that is formed has been described by Grant et al. under the name of <<egg-box model>> (Grant G. T. et al. (1973) Biological interactions between polysaccharides and divalent cations: the egg-box model, FEBS Letters, 32, 195).

There are also amidated pectins. Treatment of pectin by ammonia transforms some methyl carboxylate groups (—COOCH₃) into carboxamide groups (—CONH₂). This amidation confers novel properties to the pectins, in particular better resistance to variations in pH. Amidated pectins tend to be more tolerant to the variations in pH, and have also been studied for the manufacture of matricial tablets for colonic delivery (Wakerly Z. et al. (1997) Studies on amidated pectins as potential carriers in colonic drug delivery, Journal of Pharmacy and Pharmacology. 49, 622).

Pectin is degraded by enzymes originating from higher plants and various microorganisms (fungi, bacteria, and the like) among which bacteria from the human colonic flora. The enzymes produced by the microflora encompass a mixture of polysaccharidases, glycosidases and esterases.

Metal Cations

Divalent zinc cations from various zinc salts can be used to crosslink pectin, as well as interact with the enzyme. Examples include zinc sulfate, zinc chloride, and zinc acetate. Other di-, tri-, or polyvalent ions can be used.

Eudragit® Polymers

The coating of drug-loaded cores such as tablets, capsules, granules, pellets or crystals offers many advantages, such as higher physicochemical stability, better compliance and increased therapeutic efficiency of the active agents. Indeed, the effectiveness of a medication depends not only on the actives it contains, but also on formulation and processing.

Poly(meth)acrylates have proven particularly suitable as coating materials. These polymers, of which only a few milligrams are employed, are pharmacologically inactive, i.e., are excreted unchanged.

EUDRAGIT® is the trade name for copolymers derived from esters of acrylic and methacrylic acid, whose properties are determined by functional groups. The individual EUDRAGIT® grades differ in their proportion of neutral, alkaline or acid groups and thus in terms of physicochemical properties. The skillful use and combination of different EUDRAGIT® polymers offers ideal solutions for controlled drug release in various pharmaceutical and technical applications. EUDRAGIT® provides functional films for sustained-release tablet and pellet coatings. The polymers are described in international pharmacopeias such as Ph.Eur., USP/NF, DMF and JPE.

EUDRAGIT® polymers can provide the following possibilities for controlled drug release:
Gastrointestinal tract targeting (gastroresistance, release in the colon)
Protective coatings (taste and odor masking, protection against moisture)
Delayed drug release (sustained-release formulations).

EUDRAGIT® polymers are available in a wide range of different concentrations and physical forms (aqueous solution, aqueous dispersion, organic solution, solid substances).

The pharmaceutical properties of EUDRAGIT® polymers are determined by the chemical properties of their functional groups. A distinction is made between
poly(meth)acrylates, soluble in digestive fluids (by salt formation)

EUDRAGIT® L, S, FS and E polymers with acidic or alkaline groups enable pH-dependent release of the active agent.

Applications: from simple taste masking via resistance solely to gastric fluid, to controlled drug release in all sections of the intestine
poly(meth)acrylates, insoluble in digestive fluids

EUDRAGIT® RL and RS polymers with alkaline and EUDRAGIT® NE polymers with neutral groups enable controlled time release of the active by pH-independent swelling.

Enteric EUDRAGIT® coatings provide protection against drug release in the stomach and enable controlled release in the intestine. Targeted drug release in the gastrointestinal tract is recommended for particular applications or therapeutic strategies, for example when the agent is sparingly soluble in the upper digestive tract, or when the agent may be degraded by gastric fluid. Secondly, this dosage form is very patient-friendly as it does not stress the stomach and the number of doses of the therapeutic agent can be considerably reduced, thanks to prolonged delivery. The dominant criterion for release is the pH-dependent dissolution of the coating, which takes place in a certain section of the intestine (pH 5 to over 7) rather than in the stomach (pH 1-5). For these applications, anionic EUDRAGIT® grades containing carboxyl groups, can be mixed with each other. This makes it possible to finely adjust the dissolution pH, and thus to define the drug release site in the intestine. EUDRAGIT® L and S grades are suitable for enteric coatings. EUDRAGIT® FS 30 D is specifically used for controlled release in the colon.

Application benefits of enteric EUDRAGIT® coatings include:
pH-dependent drug release
protection of actives sensitive to gastric fluid
protection of the gastric mucosa from aggressive actives
increase in drug effectiveness
good storage stability
controlled release in the colon/GI targeting

Polycationic Polymers

In one embodiment, the pectin bead is coated with a cationic polymer. Any pharmaceutically acceptable polycationic polymer can be used, though the most common polycationic polymers are chitosan, polylysine, and polyethylene imine.

Other Types of Colonic Delivery

In addition to pectin beads, other drug delivery systems are known to provide delivery to the colon. For example, certain Eudragit® polymers are known to dissolve at the pH in the colon, and are used to formulate drug delivery systems for colonic delivery. Mucoadhesive polymers are also known to be used to deliver agents to the colon. The mucoadhesive polymers can be coated onto microparticles or nanoparticles, and when administered, and the particles reach the colon, they adhere to the mucous membrane. When adhered, the compositions can degrade over time and release the active agents. Representative bioadhesive polymers, and drug delivery systems, are described in U.S. Pat. No. 6,365,187 to Edith Mathiowitz.

Other Types of Gastro-Intestinal Delivery

In some embodiments, the delivery systems are intended to delivery the active agents to a portion of the gastrointestinal tract other than the colon, and past the stomach (where the active agents can be metabolized). For example, one can deliver agents to the distal jejunum, the proximal ileum, or directly to the ileum. Such a formulation minimizes release of the agent in the upper part of the small intestine, i.e. above the distal jejunum.

In these embodiments, the pectin beads, which specifically administer agents to the colon, would ideally not be used. Other formulations for delivery to other locations in the gastrointestinal tract are known to those of skill in the art, and intended to be part of the invention described herein. For example, Krishnamachari et al. (Int. J. Pharm, 338(1-2):238-237 (2007)) discloses microparticles with non-enzymatically degrading poly(dl-lactide-co-glycolide) (PLGA) core that deliver agents in a site specific manner to both the distal ileum and colon.

Additional Formulation Information

The formulations can conveniently be presented in unit dosage form and can be prepared by any methods well known in the art of pharmacy.

Formulations of the invention suitable for oral administration can be in the form of capsules, cachets, pills, tablets, powders, granules, pellets, or as a suspension in an aqueous or non-aqueous liquid, each containing a predetermined amount of an active agent or a combination of active agents.

In solid dosage forms of the invention for oral administration (capsules, tablets, pills, dragees, powders, granules, pellets and the like), the active agent is mixed with one or more pharmaceutically-acceptable carriers, such as (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, starch, (5) wetting agents, such as, for example, cetyl alcohol, glycerol monostearate, and non-ionic surfactants; (6) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions can also comprise buffering agents. Solid compositions of a similar type can also be employed as fillers in soft and hard-shelled gelatin capsules using such excipients as lactose or milk sugars, as well as high molecular weight polyethylene glycols and the like.

A tablet can be made by compression or molding, optionally with one or more accessory ingredients. Compressed tablets can be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets can be made by molding in a suitable machine a mixture of the powdered compound moistened with an inert liquid diluent.

The tablets, and other solid dosage forms of the pharmaceutical compositions of the present invention, such as dragees, capsules, pills, granules, can optionally be prepared with coatings and shells, such as gastro-resistant coatings and/or complementary enteric coatings to provide release of the active agent in a certain portion of the gastrointestinal tract and other coatings well known in the pharmaceutical-formulating art.

Examples of embedding compositions which can be used include polymeric substances and waxes. The active agent can also be in micro-encapsulated form, if appropriate, with one or more of the above-described excipients.

The systems with different drug release mechanisms described above can be combined in a final dosage form comprising single or multiple units. Examples of multiple units include multilayer tablets, capsules containing tablets, beads, granules, etc.

Delayed release formulations are created by coating a solid dosage form with a film of a polymer which is insoluble in the acid environment of the stomach, and soluble in the neutral environment of small intestines.

The delayed release dosage units can be prepared, for example, by coating the delivery system with a selected coating material. The agent-containing composition can be, e.g., a tablet for incorporation into a capsule, a tablet for use as an inner core in a “coated core” dosage form, or a plurality of agent-containing beads, particles or granules, for incorporation into either a tablet or capsule. Preferred coating materials include bioerodible, gradually hydrolyzable, gradually water-soluble, and/or enzymatically degradable polymers, and can be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon.

Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit®. (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), Eudragit® NE, RL and RS (water-insoluble polymers having different degrees of permeability and expandability) and Eudragit FS30D a tercopolymer of methacrylic acid, methyl acrylate and methylmethacrylate; vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials can also be used. Multi-layer coatings using different polymers can also be applied. The preferred coating weights for particular coating materials can be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition can include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates can also be used. Pigments such as titanium dioxide can also be used. Small quantities of an anti-foaming agent, such as a silicone (e.g., simethicone), can also be added to the coating composition.

Alternatively, a delayed release tablet can be formulated by dispersing the agent within a matrix of a suitable material such as a hydrophilic polymer or a fatty compound. The hydrophilic polymers can be comprised of polymers or copolymers of cellulose, cellulose ester, acrylic acid, methacrylic acid, methyl acrylate, ethyl acrylate, and vinyl or enzymatically degradable polymers or copolymers as described above. These hydrophilic polymers are particularly useful for providing a delayed release matrix. Fatty compounds for use as a matrix material include, but are not limited to, waxes (e.g. carnauba wax) and glycerol tristearate. Once the active agent is mixed with the matrix material, the mixture can be compressed into tablets.

These dosage forms can be administered to humans and other animals for therapy by any suitable route of administration.

Actual dosage levels of the active agents in the pharmaceutical compositions of this invention can be varied so as to obtain an effective removal of any residual antibiotic or chemical or toxin in the intestinal tract, for a particular patient, composition, and mode of administration, without being toxic to the patient.

Active Agents

The active agents described herein are not limited to a particular molecular weight. They can be entire bacteriophages, phage proteins/peptides, other antimicrobial peptides, and/or other large molecules (i.e., those with a molecular weight above about 1000) or small molecules (i.e., peptides and aptamers with a molecular weight below about 1000). Examples of suitable types of compounds include antibodies, antibody fragments, enzymes, peptides and oligonucleotides. The proteins and peptides can include only naturally-occurring amino acids, or can include non-naturally occurring (modified) amino acids. Similarly, the oligonucleotides can include both naturally-occurring and modified bases.

The active agent can be introduced into the drug delivery system as a powder, a solution, a suspension, or complexed with a solubilizing agent, such as a cyclodextrin or any other suitable compound.

Some of the active agents described herein can be administered in the form of prodrugs. Prodrugs have been widely studied for the colonic targeting of various active agents (such as steroid and non-steroid anti-inflammatory agents, and spasmolytics). These systems are based on the capacity of the enzymes produced by the colonic flora to act on the prodrugs to release the active form of the active agent.

The prodrugs can be based on the action of bacterial azoreductases, so that the active agents are targeted to the colon with the drug delivery systems described herein, and the active agents are formed by reaction of the prodrug with a bacterial azoreductase, which provides a dual mechanism for ensuring that the agents are administered to the colon. Representative chemistry for forming such prodrugs is described, for example, in Peppercorn M. A. et al. (1972) The role of intestinal bacteria in the metabolism of salicylazosulfapyridin, The Journal of Pharmacology and Experimental Therapeutics, 181, 555 and 64, 240.

Another approach consists in using bacterial hydrolases such as glycosidases and polysaccharidases (Friend D. R. (1995) Glycoside prodrugs: novel pharmacotherapy for colonic diseases, S.T.P. Pharma Sciences, 5, 70; Friend D. R. et al. (1984) A colon-specific drug-delivery system based on drug glycosides and the glycosidases of colonic bacteria, Journal of Medicinal Chemistry, 27, 261; Friend D. R. et al. (1985) Drug glycosides: potential prodrugs for colon-specific drug delivery, Journal of Medicinal Chemistry, 28, 51; and Friend D. R. et al. (1992) Drug glycosides in oral colon-specific drug delivery, Journal of Controlled Release, 19, 109). Prodrugs have thus been developed by coupling, for example, sugar with steroids (glucose, galactose, cellobiose, dextrane (international application WO 90/09168)), cyclodextrins Hirayama F. et al. (1996) In vitro evaluation of Biphenylyl Acetic Acid-beta-Cyclodextrin conjugates as colon-targeting prodrugs: drug release behavior in rat biological media, Journal of Pharmacy and Pharmacology, 48, 27).

a) Bacteriophage

Bacteria have natural enemies, particularly, host-specific viruses, known as bacteriophages or phages, which infect and kill bacteria in the natural environment. Such bacteriophages generally have small compact genomes and bacteria are their exclusive hosts. Many known bacteria are host to a large number of bacteriophages that have been described in the literature, any of which can be used in the invention described herein.

Bacteriophages successfully infect and inhibit or kill host bacteria, targeting a variety of normal host metabolic and physiological traits, some of which are shared by all bacteria, pathogenic and nonpathogenic alike. The term “pathogenic” as used herein denotes a contribution to or implication in disease or a morbid state of an infected organism.

Any bacteriophage which can inhibit pathogenic bacteria, whether or not it also inhibits non-pathogenic bacteria, is suitable for use in the invention described herein.

b) Phage Proteins

Antimicrobial phage proteins or peptides can be isolated from phage, or sequenced along according to a structure elucidated from the phage, and used in the invention described herein.

Some antimicrobial phage proteins are already known, and can be used in the compositions and methods described herein. Examples include those described in U.S. Pat. No. 6,432,444 by Fischetting and Loomis, the contents of which are hereby incorporated by reference. Others are determined, for example, by identifying a bacteriophage nucleic acid coding region encoding a product active on an essential bacterial target by identifying a nucleic acid sequence encoding a gene product which provides a bacteria-inhibiting function when the bacteriophage infects a host bacterium. This can be accomplished, for example, by identifying and elucidating the molecular mechanisms by which suitable phages interfere with host bacterial metabolism (i.e., identifying an active phage protein). The identified (and isolated) protein can be used, rather than the entire bacteriophage.

Whether the phage blocks bacterial RNA transcription or translation, or attacks other important metabolic pathways, such as cell wall assembly or membrane integrity, the basic blueprint for a phage's bacteria-inhibiting ability is encoded in its genome and can be unlocked using bioinformatics, functional genomics, and proteomics. Sequence information from the genomics of bacteriophage can be used to identify novel antimicrobial proteins/peptides which can be used as active agents to treat and/or prevent colonic bacterial infections.

In one embodiment, the phage protein may be an enzyme that attacks the major polymer of the bacterial wall, peptidoglycan, by binding to it and cleaving bonds which are required for its stability. Such enzymes, called lysins or bacterial cell wall hydrolases, can be used in an isolated form to lyse sensitive Gram-positive bacteria from the outside. Such an activity has been described in Loeffler, J. M., Nelson, D. and Fischetti, V. A., 2001, “Rapid killing of Streptococcus pneumonia with a bacteriophage cell wall hydrolase” Science vol. 294, 2170-2172; Fischetti, V. A., 2005, “Bacteriophage lytic enzymes: novel anti-infectives” Trends in Microbiology vol. 13, 491-496; cited here for reference only.

When identifying and elucidating the mechanisms by which the phages are active, as well as the correspondingly active phage proteins/peptides, one typically must identify the bacteria-inhibiting phage open reading frames (“ORF”s) and corresponding peptides/proteins that can be used as active agents (i.e., based on the amino acid sequence and secondary structural characteristics of the ORF products).

In one embodiment, at least one recombinant phage ORF(s) is expressed in a bacterial host, and inhibition analysis of that host is performed Inhibition following expression of the phage ORF is indicative that the product of the ORF is active on an essential bacterial target. In one aspect, a plurality of phage ORFs can be expressed in a single bacterium, to identify a plurality of phage proteins/peptides that inhibit the bacteria. In another aspect, a plurality of bacteria are evaluated, where one ORF is expressed in each bacterium, to identify proteins/peptides that are selectively inhibitory for one type of bacteria over another (ideally, inhibitory for harmful bacteria over helpful bacteria). In yet another aspect, a plurality of bacteria is used in conjunction with a plurality of ORFs expressed in at least one or in all of the plurality of bacteria.

The inhibitory function of the phage ORF can be confirmed, for example, by expressing and isolating the ORF product, and determining whether the isolated product has the desired activity. That is, by inhibiting a desired bacterium following expression of a phage ORF, one confirms that the product has the desired activity. Such approaches are routinely understood and accomplished by those of skill in the art using standard techniques.

In order to select desired antimicrobial phage proteins/peptides, it can be desired to perform the analysis of the ORF of the phage using a pathogenic bacterial species, rather than a helpful bacterial species. That way, the protein/peptide is sure to inhibit the harmful bacteria, even if it also inhibits helpful bacteria.

In some cases, however, one may wish to also use phage with non-pathogenic host bacteria. Accordingly, phage proteins/peptides that are effective against the beneficial (non-pathogenic) bacteria can be compared with those that are effective against pathogenic bacteria. Proteins/peptides active at least against the pathogenic bacteria, and ideally, not against the non-pathogenic bacteria, can be identified.

In some embodiments, the phage proteins/peptides will function against known targets in the bacteria, and in others, the inhibition occurs without knowledge of the precise mechanism.

Identification of the bacterial target can involve identification of a phage-specific site of action. This can involve a newly identified target, or a target where the phage site of action differs from the site of action of a previously known antibacterial agent or inhibitor. For example, phage T7 genes 0.7 and 2.0 target the host RNA polymerase, which is also the cellular target for the antibacterial agent, rifampin.

Stating that an agent or compound is “active on” a particular cellular target, such as the product of a particular gene, means that the target is an important part of a cellular pathway which includes that target and that the agent acts on that pathway. Thus, in some cases the agent may act on a component upstream or downstream of the stated target, including on a regulator of that pathway or a component of that pathway.

By “essential”, in connection with a gene or gene product, is meant that the host cannot survive without, or is significantly growth compromised, in the absence depletion, or alteration of functional product. An “essential gene” is thus one that encodes a product that is beneficial, or preferably necessary, for cellular growth in vitro in a medium appropriate for growth of a strain having a wild-type allele corresponding to the particular gene in question. Therefore, if an essential gene is inactivated or inhibited, that cell will grow significantly more slowly, preferably less than 20%, more preferably less than 10%, most preferably less than 5% of the growth rate of the uninhibited wild-type, or not at all, in the growth medium. Preferably, in the absence of activity provided by a product of the gene, the cell will not grow at all or will be non-viable, at least under culture conditions similar to the in vivo conditions normally encountered by the bacterial cell during an infection. For example, absence of the biological activity of certain enzymes involved in bacterial cell wall synthesis can result in the lysis of cells under normal osmotic conditions, even though protoplasts can be maintained under controlled osmotic conditions. In the context of the invention, essential genes are generally the preferred targets of antimicrobial agents. Essential genes can encode target molecules directly or can encode a product involved in the production, modification, or maintenance of a target molecule.

c) Peptide Phage Display Libraries

In an analogous fashion, one can identify phage peptides that bind particular receptors using peptide phage display, starting with a phage display library. Phage display technology is described, for example, in Phage Display of Peptides and Proteins: A Laboratory Manual; Edited by Brian K. Kay et al. Academic Press San Diego, 1996, the contents of which are hereby incorporated by reference for all purposes.

Phage peptide libraries typically include numerous different phage clones, each expressing a different peptide, encoded in a single stranded DNA genome as an insert in one of the coat proteins. In an ideal phage library the number of individual clones would be 20ⁿ, where “n” equals the number of residues that make up the random peptides encoded by the phage. For example, if a phage library was screened for a seven residue peptide, the library in theory would contain 20⁷ (or 1.28×10⁹) possible 7 residue sequences. Therefore, a 7-mer peptide library should contain approximately 10⁹ individual phage.

Methods for preparing libraries containing diverse populations of various types of molecules such as antibodies, peptides, polypeptides, proteins, and fragments thereof are known in the art and are commercially available (see, for example, Ecker and Crooke, Biotechnology 13:351360 (1995), and the references cited therein, the contents of each of which is incorporated herein by reference for all purposes). One example of a suitable phage display library is the Ph.D.7 phage display library (New England BioLabs Cat #8100), a combinatorial library consisting of random peptide 7-mers. The Ph.D.7 phage display library consists of linear 7-mer peptides fused to the pIII coat protein of M13 via a GlyGlyGlySer flexible linker. The library contains 2.8 times 10⁹ independent clones and is useful for identifying targets requiring binding elements concentrated in a short stretch of amino acids.

Phage clones displaying peptides that are able to bind to the targets are selected from the library. The sequences of the inserted peptides are deduced from the DNA sequences of the phage clones. This approach is particularly desirable because no prior knowledge of the primary sequence of the target protein is necessary, epitopes represented within the target, either by a linear sequence of amino acids (linear epitope) or by the spatial juxtaposition of amino acids distant from each other within the primary sequence (conformational epitope) are both identifiable, and peptidic mimotopes of epitopes derived from non-proteinaceous molecules such as lipids and carbohydrate moieties can also be generated.

A library of phage displaying potential binding peptides can be incubated with immobilized targets to select clones encoding recombinant peptides that specifically bind the immobilized targets. The phages can be amplified after various rounds of biopanning (binding to the immobilized targets) and individual viral plaques, each expressing a different recombinant protein, or binding peptide, can then be expanded to produce sufficient amounts of peptides to perform a binding assay.

Phage selection can be conducted according to methods known in the art and according to manufacturers' recommendations. “Target” proteins, identified as being suitable targets for inhibiting bacterial growth, can be coated overnight onto high binding plastic plates or tubes in humidified containers. In a first round of panning, approximately 2×10¹¹ phage can be incubated on the protein-coated plate for 60 minutes at room temperature while rocking gently. The plates can then be washed using standard wash solutions. The binding phage can then be collected and amplified following elution using the target protein. Secondary and tertiary pannings can be performed as necessary.

Following the last screening, individual colonies of phage-infected bacteria can be picked at random, the phage DNA isolated and then subjected to dideoxy sequencing. The sequence of the displayed peptides can be deduced from the DNA sequence.

Once identified as being antimicrobial phage peptides, the peptides can be produced using any of a variety of known methods and delivered to the colon using the drug delivery systems described herein.

d) Antimicrobial Aptamers

As used herein, oligonucleotides typically have a molecular weight less than about 10,000 (about 30-mer or less). High throughput screening methods such as SELEX can be used to identify oligonucleotides that bind particular bacterial receptors, and thus inhibit bacterial growth, starting with a library including random oligonucleotides, typically with sizes less than about 100mers (also referred to as “aptamers”). The SELEX method is described in U.S. Pat. No. 5,270,163 to Gold et al. Briefly, a candidate mixture of single stranded nucleic acids with regions of randomized sequence can be contacted with the targets and those nucleic acids having an increased affinity to the targets can be partitioned from the remainder of the candidate mixture. The partitioned nucleic acids can be amplified to yield a ligand enriched mixture.

The SELEX process can be used to screen such oligonucleotide libraries (including DNA, RNA and other types of genetic material, and also including natural and non-natural base pairs) for aptamers that have suitable binding properties to suitable bacterial targets, and other assays can be used to determine the antimicrobial effects of the aptamers.

e) Antimicrobial Peptides

Antimicrobial peptides (AMPs) are part of a host's innate immune system in many organisms and serve as the first line of defense against microbial invasion. Highly stable to adverse conditions, AMPs bind semi-selectively to microbial cell surfaces and exert their antimicrobial activity through membrane disruption. Given their ability to bind to multiple target microbes, an array consisting of multiple AMPs can potentially be capable of detecting a higher number of target species than an array with a corresponding number of antibodies. Furthermore, the predicted stability of the AMPs within these arrays is expected to improve operational and logistical constraints over current antibody-based systems. The AMP-based arrays differ from standard peptide arrays in that some or all components are naturally occurring (or derivatives of molecules produced in nature) and may have defined secondary structures, unlike combinatorially derived libraries. Most importantly, as many AMPs have overlapping specificities, the pattern of differences in binding affinities can be used for identification.

The antimicrobial peptides include naturally-occurring anti-microbial peptides, as well as chimeric peptides, genetic variants, and synthetic mimics.

One class of AMPs is comprised of linear peptides that naturally fold to form two helical domains: a strongly basic helical region and a hydrophobic helix separated by a short hinge region. Magainins and other amphipathic alpha-helical AMPs are unstructured in solution, but become helical upon interaction with target membranes. Because of its stability and ability to bind to multiple bacterial species magainin I (GIGKFLHSAGKFGKAFVGEIMKS) may be used as a recognition molecule for incorporation into an array-based sensor for detection of pathogenic bacteria.

In general, use of multiplexed antimicrobial peptides and antibiotics for target detection may possess the following advantages over standard antibody-based detection techniques:

Not all interactions between AMPs and membranes of target organisms are fully characterized, but they have been demonstrated to occur in the absence of specific receptors. Cationic peptides are thought to preferentially interact with negatively charged phospholipids on bacterial and fungal membranes, with only marginal activity against zwitterionic phospholipids. Most cationic peptides therefore exhibit selective toxicity for bacterial, fungal, and protozoan targets, rather than mammalian ones, and may preferentially interact with Gram-negative bacteria over Gram-positive species. On the other hand, AMPs with hydrophobic segments (e.g., melittin, alamethicin) are highly toxic to mammalian cells but also bind with high affinity to bacterial membranes.

The mechanism of membrane disruption is believed to occur by formation of either “carpets” or channels. The “carpet” mechanism involves binding of charged (typically cationic) amino acids to headgroups of membrane phospholipids or lipopolysaccharide. After initial binding, AMPs aggregate to form a “carpet,” with helices or .beta.-sheets oriented parallel to the membrane surface. Upon rotation of the AMP chains, hydrophobic side chains are inserted into the membrane, disrupting lipid packing, or alternatively, creating a toroidal pore. Channel formation, on the other hand, involves insertion of the peptide backbone into the membrane, rather than the side chains. After insertion of the peptide backbone into the membrane, AMPs aggregate to form a barrel-like structure with a central aqueous channel. A feature of both mechanisms is the requirement for multiple AMPs and for peptide-peptide interactions. The attached claims are not intended as requiring these or any other mechanisms.

Since antimicrobial peptides typically recognize target pathogens by interacting with the microbial cell membranes, and most peptide-membrane and antibiotic-membrane interactions do not involve specific receptors, but rather invariant components of the cell surface, binding is therefore semi-selective. Thus, each peptide can bind to multiple microbial species with differing affinities. However, certain peptides function by inhibiting specific enzymes or receptors, which in some embodiments can offer selectivity for one type of bacteria (ideally, a pathogenic bacteria) over another type of bacteria (ideally, a non-pathogenic, or helpful bacteria).

Anti-microbial peptides within the scope of the present invention are indolicidin, apidaecin, bacteriocin, alpha-helical clavanin, magainin, cecropin, andropin, histatin, beta-pleated sheet bacteriocin dodecapeptide, tachyplesin, protegrin, defensin, beta-defensin, alpha-defensin (Miyasaki and Lehrer, 1998, Intl. J. Antimicrobial Agents 9:270-272); alexomycin (Marshel and Jones, 1999, Diagn. Micrbiol. Infect. Dis. 33:183-184); nisin, ranalexin, buforin (Giacometti et al., 1999, Peptides 30:1266). Derivatives and analogues of such peptides are likewise within the scope of the present invention. The relatively simple structure of bacteriocidal peptides lends itself to designing a peptide with increased anti-microbial activities and decreased host cell toxicity.

More specifically, buforin, nisin and cecropin have antimicrobial effects on Escherichia. coli, Shigella dysenteriae, Salmonella typhimurium, Streptococcus pneumoniae, Staphylococcus aureus, and Pseudomonas aeruginosa.

Magainin and ranalexin have antimicrobial effects on the same organsims, and in addition has such effects on Candida albicans, Cryptococcus neoformans, Candida krusei, and Helicobacter pylori.

Protegrin has antimicrobial effects on Neisseria gonorrhoeae, Chlamydia trachomatis and Haemophilus influenzae.

Alexomycin has antimicrobial effects on Campylobacter jejuni, Moraxella catarrhalis and Haemophilus influenzae.

Alpha-defensin and beta-pleated sheet defensin have antimicrobial effects on Streptococcus pneumoniae. Additional antimicrobial peptides include Plectasin and its derivatives. Plectasin is a defensin derived from fungus (see Mygind, 2005, Nature 437, 975. Plectasin is patented by Novozymes, which has also made highly active derivatives, including NZ2114

Other antimicrobial peptides are described in U.S. Patent Application Publication No. 20050215481. The antimicrobial peptides described in U.S. Patent Application Publication No. 20050215481 include a central fragment with a relatively conserved amino acid sequence and alternating basic amino acid residues and hydrophobic amino acid residues at the N-terminus and C-terminus sides of the above central fragment. Thereby, the secondary structure of the total peptide is stabilized and the peptide is able to penetrate into microbial cells and act against the microorganisms.

The antimicrobial peptides can be analogs of the peptides described above, which are amidated at C-terminus. The antimicrobial peptides whose C-terminus is amidated typically show improved antimicrobial activities against Gram-positive and Gram-negative bacteria, and fungi.

The antimicrobial peptides can further be reverse sequences of known antimicrobial peptides. Such reverse peptides include those described in U.S. Pat. No. 5,519,115.

The '115 patent teaches that, by reversing the sequence of antimicrobial peptides such as Magainins 1 and 2, the resulting reverse peptides had specific antimicrobial activity, and/or resistance to proteolytic degradation. However, reversal of the sequence of some peptides sometimes results in sacrifices in the activity of the peptides. In some cases, additional modifications to the peptides can result in improved activity. For example, in the '115 patent, the sequence of Magainin 2 was amended by substituting the Ser at position 8 with a Glu, which caused the resulting peptide to have a substantial reduction in the extent of proteolysis.

Mixtures of two or more of antimicrobial peptides can provide a broad spectrum of protection which cannot be realized by using one antimicrobial peptide alone. These compounds can be provided as a mixture having broad spectrum activity against pathogenic bacteria, while at the same time having increased overall resistance to proteolysis. These mixtures can be provided by producing, collecting and intermixing two or more antimicrobial peptides and/or by engineering a synthetic gene or genes to co-express these peptides in transformed host cells.

For example, the reverse antimicrobial peptide can be active against at least one microbial pathogen, and ideally has at least about an equal degree of resistance to proteolytic degradation when compared to a peptide of the identical sequence in the opposite order. Examples include reverse Magainins, reverse PGLc, reverse Pl's, reverse Cecropins, reverse Sarcotoxins, reverse Bombinins, reverse XPFs, reverse Thionins, reverse Defensins, reverse Melittins, and reverse PGLa.

A variety of silent amino acid substitutions, additions, or deletions can be made in the antimicrobial peptides described herein, so long as they do not significantly alter the fragments' antimicrobial activity.

The peptides can be generated in vitro using peptide synthesizers, or can be produced in cell culture by inserting an appropriate sequence in a cell.

The proteins and/or peptides can also be produced from recombinant sources, from genetically altered cells implanted into animals, from tumors, and from cell cultures as well as other sources. Antimicrobial peptides can be isolated from body fluids including, but not limited to, mucosa. Recombinant techniques include gene amplification from DNA sources using the polymerase chain reaction (PCR), and gene amplification from RNA sources using reverse transcriptase/PCR.

Identifying Peptides which Specifically Affect Pathogenic Bacteria

U.S. Patent Application Publication No. 20060281074 discloses an assay method for identifying antimicrobial peptides which bind to certain targets, and not others, which are therefore selective for certain bacteria over other bacteria. This type of assay can be used to identify antimicrobial peptides (as well as phage proteins, antimicrobial peptides, and aptamers) which bind to targets in pathogenic (harmful) bacteria, and which do not bind (or bind with less affinity) to targets in non-pathogenic (helpful) bacteria.

In this method, a plurality of antimicrobial peptides is bound to one or more substrates. At least one of the antimicrobial peptides is suspected of having one or more biological targets affinity bound to it. One then detects which antimicrobial peptide(s) have bound the biological targets, and this information is used to generate a binding pattern. The biological target is then identified based on the binding pattern. Analogous assays can ideally be performed with a plurality of aptamers.

f) Antibiotics which are Toxic if Administered Systemically

There are a variety of antibiotics which are toxic if administered systemically, for example, polypeptide antibiotics such as colistin (Colimycin), aminoglycosides, and the like. Although efforts have been made to orally administer some of these agents to provide selective decontamination in the colon, their systemic toxicity at relatively high doses minimized their effectiveness. For example, one researcher found that in human volunteers, only high dosages of Colimycin were effective at achieving selective decontamination. However, these high doses were very poorly tolerated at the gastric level (van Saene J J, van Saene H K, Tarko-Smit N J, Beukeveld G J. Enterobacteriaceae suppression by three different oral doses of polymyxin E in human volunteers. Epidemiol Infect. 1988 June; 100(3):407-17.). The present invention can provide higher dosages of antimicrobial agents that are toxic if administered systemically at these higher dosages, but delivering them specifically to the colon site. This allows one to deliver high doses of colimycin and other like agents to the colon without having the gastric side effects.

In one aspect of this embodiment, one can simultaneously treat a patient with beneficial bacteria, such as E. coli, lactobacilli or bifidobacteria, or fungi, such as Saccharomyces to provide selective decontamination by increasing the population of beneficial bacteria. In this aspect, the beneficial effect of the good bacteria, coupled with the agent specifically administered to selectively eliminate the pathogenic bacteria, can be advantageous.

g) Antibodies

Antibodies can be generated that:

a) bind to relevant targets on harmful bacteria, but not on analogous targets on helpful bacteria,

b) are effective at lysing bacterial cell walls, or

c) are effective at binding to or otherwise inactivating bacterial toxins.

Polyclonal antibodies can be used, provided their overall effect is decreased bacterial concentration. However, monoclonal antibodies are preferred. Humanized (chimeric) antibodies can be even more preferred.

Antibodies, in particular, monoclonal antibodies (mAbs) have been developed which are themselves antimicrobial, or which activate the immune system against a bacterial infection. The antibodies can be extremely specific.

Antibody Preparation

The term “antibody” refers to a polypeptide substantially encoded by an immunoglobulin gene or immunoglobulin genes, or fragments thereof, that specifically binds and recognizes an analyte (antigen, in this case a bacterial target). Immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit includes a tetramer. Each tetramer is composed of two identical pairs of polypeptide chains, each pair having one “light” (about 25 kD) and one “heavy” chain (about 50-70 kD). The N-terminus of each chain has a variable region of about 100 to 110 or more amino acids primarily responsible for antigen recognition. The terms “variable light chain” (or “VL”) and “variable heavy chain” (or “VH”) refer to these light and heavy chains, respectively.

Antibodies exist, for example, as intact immunoglobulins or as a number of well characterized antigen-binding fragments produced by digestion with various peptidases. For example, pepsin digests an antibody below the disulfide linkages in the hinge region to produce an F(ab′)₂ fragment, a dimer of Fab which itself is a light chain joined to VH-CH1 by a disulfide bond. The F(ab′)2 fragment can be reduced under mild conditions to break the disulfide linkage in the hinge region, thereby converting the F(ab′)2 dimer into an Fab′ monomer. The Fab′ monomer is essentially an Fab with part of the hinge region (see Fundamental Immunology, Third Edition, W. E. Paul (ed.), Raven Press, N.Y. (1993), the contents of which are hereby incorporated by reference). While various antibody fragments are defined in terms of the digestion of an intact antibody, one of ordinary skill in the art will appreciate that such fragments can be synthesized de novo either chemically or by using recombinant DNA methodology. Thus, the term antibody, as used herein, also includes antibody fragments, such as a single chain antibody, an antigen binding F(ab′) 2 fragment, an antigen binding Fab′ fragment, an antigen binding Fab fragment, an antigen binding Fv fragment, a single heavy chain or a chimeric (humanized) antibody. Such antibodies can be produced by modifying whole antibodies or synthesized de novo using recombinant DNA methodologies.

The relevant targets identified from harmful bacteria (including fragments, derivatives, and analogs thereof) can be used as an immunogen to generate antibodies which immunospecifically bind such immunogens. Such antibodies include but are not limited to polyclonal antibodies, monoclonal antibodies, chimeric antibodies, single chain antibodies, antigen binding antibody fragments (e.g., Fab, Fab′, F(ab′)2, Fv, or hypervariable regions), and mAb or Fab expression libraries. In some embodiments, polyclonal and/or monoclonal antibodies to these targets, or the fragments, derivatives and/or analogs thereof are produced. In yet other embodiments, fragments of the targets that are identified as immunogenic are used as immunogens for antibody production.

Various procedures known in the art can be used to produce polyclonal antibodies. Various host animals (including, but not limited to, rabbits, mice, rats, sheep, goats, camels, and the like) can be immunized by injection with the antigen, fragment, derivative or analog. Various adjuvants can be used to increase the immunological response, depending on the host species. Such adjuvants include, for example, Freund's adjuvant (complete and incomplete), mineral gels such as aluminum hydroxide, surface active substances such as lysolecithin, pluronic polyols, polyanions, peptides, oil emulsions, keyhole limpet hemocyanins, dinitrophenol, and other adjuvants, such as BCG (bacille Calmette-Guerin) and Corynebacterium parvum.

Any technique that provides for the production of antibody molecules by continuous cell lines in culture can be used to prepare monoclonal antibodies directed toward the bacterial target, fragments thereof or binding portions thereof. Such techniques include, for example, the hybridoma technique originally developed by Kohler and Milstein (see, e.g., Nature 256:495-97 (1975)), the trioma technique (see, e.g., Hagiwara and Yuasa, Hum. Antibodies Hybridomas 4:15-19 (1993); Hering et al., Biomed. Biochim. Acta 47:211-16 (1988)), the human B-cell hybridoma technique (see, e.g., Kozbor et al., Immunology Today 4:72 (1983)), and the EBV-hybridoma technique to produce human monoclonal antibodies (see, e.g., Cole et al., In: Monoclonal Antibodies and Cancer Therapy, Alan R. Liss, Inc., pp. 77-96 (1985)). Human antibodies can be used and can be obtained by using human hybridomas (see, e.g., Cote et al., Proc. Natl. Acad. Sci. USA 80:2026-30 (1983)) or by transforming human B cells with EBV virus in vitro (see, e.g., Cole et al., supra).

“Chimeric” or “humanized” antibodies (see, e.g., Morrison et al., Proc. Natl. Acad. Sci. USA 81:6851-55 (1984); Neuberger et al., Nature 312:604-08 (1984); Takeda et al., Nature 314:452-54 (1985)) can also be prepared. Such chimeric antibodies are typically prepared by splicing the non-human genes for an antibody molecule specific for antigen together with genes from a human antibody molecule of appropriate biological activity. It can be desirable to transfer the antigen binding regions (e.g., Fab′, F(ab′)2, Fab, Fv, or hypervariable regions) of non-human antibodies into the framework of a human antibody by recombinant DNA techniques to produce a substantially human molecule. Methods for producing such “chimeric” molecules are generally well known and described in, for example, U.S. Pat. Nos. 4,816,567; 4,816,397; 5,693,762; and 5,712,120; PCT Patent Publications WO 87/02671 and WO 90/00616; and European Patent Publication EP 239 400 (the disclosures of which are incorporated by reference herein). Alternatively, a human monoclonal antibody or portions thereof can be identified by first screening a cDNA library for nucleic acid molecules that encode antibodies that specifically bind to the bacterial targets or fragments or binding domains thereof according to the method generally set forth by Huse et al. (Science 246:1275-81 (1989)), the contents of which are hereby incorporated by reference. The nucleic acid molecule can then be cloned and amplified to obtain sequences that encode the antibody (or antigen-binding domain) of the desired specificity. Phage display technology offers another technique for selecting antibodies that bind to the bacterial targets, fragments, derivatives or analogs thereof and binding domains thereof (See, e.g., International Patent Publications WO 91/17271 and WO 92/01047; Huse et al., supra.) Techniques for producing single chain antibodies (see, e.g., U.S. Pat. Nos. 4,946,778 and 5,969,108) can also be used. An additional aspect of the invention utilizes the techniques described for the construction of a Fab expression library (see, e.g., Huse et al., supra) to allow rapid and easy identification of monoclonal Fab fragments with the desired specificity for antigens, fragments, derivatives, or analogs thereof.

Antibodies that contain the idiotype of the molecule can be generated by known techniques. For example, such fragments include but are not limited to, the F(ab′)2 fragment which can be produced by pepsin digestion of the antibody molecule, the Fab′ fragments which can be generated by reducing the disulfide bridges of the F(ab′)2 fragment, the Fab fragments which can be generated by treating the antibody molecule with papain and a reducing agent, and Fv fragments. Recombinant Fv fragments can also be produced in eukaryotic cells using, for example, the methods described in U.S. Pat. No. 5,965,405 (the disclosure of which is incorporated by reference herein).

Antibody screening can be accomplished by techniques known in the art (e.g., ELISA (enzyme-linked immunosorbent assay)). In one example, antibodies that recognize a specific domain of an antigen can be used to assay generated hybridomas for a product which binds to polypeptides containing that domain. Antibodies specific to a domain of an antigen are also provided.

Antibodies against the relevant targets (including fragments, derivatives and analogs and binding domains thereof) can be administered in the compositions described herein, and delivered to the colon using the drug delivery systems described herein.

Small amounts of humanized antibody can be produced in a transient expression system in CHO cells to establish that they bind to bacterial cells expressing the relevant target. Stable cell lines can then be isolated to produce larger quantities of purified material.

Screening Methods for Identifying Antimicrobial Antibodies

High throughput monoclonal antibody assays can be used to determine the binding affinities of the antibodies to targets which inhibit bacterial growth, bacterial toxins and/or which antibodies lyse bacterial cell walls.

Similar screening methods can be used to identify other classes of compounds useful in the methods described herein. Combinatorial libraries of compounds, for example, phage display peptide libraries and oligonucleotide (aptamer) libraries can be screened. Compounds that bind to the targets can be identified, for example, using competitive binding studies.

h) Agents which Bind to or Otherwise Inactive Bacterial Toxins

As discussed above, antibodies can be developed which bind to or otherwise inactivate bacterial toxins. There are other active agents which are known to bind to or otherwise inactivate bacterial toxins, including the polymer Tolevamer™. These agents can be particularly useful in helping to minimize damage from bacterial toxins, while other agents can be used to treat the bacterial infection itself.

### II. Screening Methods

Various screening methods can be used to determine the ability of active agents to inhibit microbial growth.

In some embodiments, the agents function by lysing bacterial cell walls, so the screening methods simply evaluate the ability of the agents to inhibit bacterial growth by lysing bacterial cell walls. In other embodiments, the compounds act by binding to and interacting with various receptors in a bacterial cell, or other suitable bacterial targets. Although many compounds can bind to the relevant targets present on certain (ideally pathogenic) bacteria, the mere fact that they bind the targets does not determine their ultimate antimicrobial properties. The screening methods can be used to determine the ultimate effect of the active agents, once bound, on the bacteria.

Various screening methods can thus be used to determine the activity of compounds bound to the targets. Examples of suitable screening methods include measuring bacterial growth, whether applied to a single bacterial population or to a series of bacterial populations. The active agents can be evaluated using in vitro assays to determine their biological activity. These assays are familiar to those skilled in the art. When it is desired that the active agents inhibit pathogenic bacteria, but not non-pathogenic bacteria, the agents can be screened against colonies of appropriate bacteria.

The biological activity of the active agents may also be tested in vivo. In such assays, an animal species which is tolerant to the target bacterial species is used. The target organisms are inoculated orally, or by gastric gavage if necessary. Then the drug delivery system containing the active agent or a placebo is given; the number of target organisms present in the colonic flora is counted in the feces or in the intestinal content if the animal is sacrified and the differences in counts between the animals treated with the placebo and those treated with the test system are measured. Statistical analysis is performed on theses differences to judge for significance. First, for the purpose of screeding a large number of potentially active molecules with designed delivery systems, small rodents such as mice or rats are used and then larger animal with intestinal physiology more similar to that of humans (such as pigs, piglets or minipigs) are used in the same manner to select the best combination of active and delivery system.

In another type of in vivo assay, animals which are susceptible to the target organisms (for instance guinea pigs for Clostridium difficile) can be used. Then the target organisms are inoculated first and then the selected active product formulation. The appearance or symptoms or death are recorded and tested for significance between animals with and without the treatment. Suitable binding assays are known to those of skill in the art.

Many examples of such assays can be find in the literature using either rodents (see for examples Paterson D L, Stiefel U, Donskey C J. Effect of a selective decontamination of the digestive tract regimen including parenteral cefepime on establishment of intestinal colonization with vancomycin-resistant Enterococcus spp. and Klebsiella pneumoniae in mice Antimicrob Agents Chemother. 2006 July; 50(7):2537-40.; Manson W L, Dijkema H, Klasen H J. Alteration of wound colonization by selective intestinal decontamination in thermally injured mice. Burns. 1990 June; 16(3):166-8; van der Waaij D. Selective decontamination of the digestive tract with oral aztreonam and temocillin. Rev Infect Dis. 1985 November-December; 7 Suppl 4:S628-34; Chang T W, Bartlett J G, Gorbach S L, Onderdonk A B. Clindamycin-induced enterocolitis in hamsters as a model of pseudomembranous colitis in patients. Infect Immun. 1978 May; 20(2):526-9.) or larger animals such as horses, piglets or pigs (see respectively for examples: Staempfli H R, Prescott J F, Carman R J, McCutcheon L J. Use of bacitracin in the prevention and treatment of experimentally-induced idiopathic colitis in horses. Can J Vet Res. 1992 July; 56(3):233-6; Mandel L, Talafantová M, Trebichavský I, Trávnícek J, Koukal M. Selective decontamination, induced colonization resistance and connected immunological changes in piglets. Folia Microbiol (Praha). 1985; 30(3):312-8; Van der Waaij L A, Messerschmidt O, Van der Waaij D. A norfloxacin dose finding study for selective decontamination of the digestive tract in pigs. Epidemiol Infect. 1989 February; 102(1):93-103.)

### III. Methods for Preparing the Pectin Beads

Pectin beads can be prepared using methods known to those of skill in the art, including by mixing the active agent in a pectin solution, and gelification of the pectin anionic moieties by a divalent, trivalent, or polyvalent cation such as divalent zinc in the form of acetate solution, for example.

This is typically done by stiffing a solution, suspension or dispersion of the active agent and pectin, adjusting the pH of the solution if necessary and adding this solution dropwise to a gelling solution containing a cationic ion (such as zinc acetate salt solution), under agitation.

The technologies for adding the pectin solution dropwise to the gelling solution are known to those of skill in the art; it includes the multi nozzle system from Nisco Engineering AG or any other relevant technology to produce drops from a pectin solution.

The pectin drops undergo a gelification process, ideally during a predetermined time to obtain the best encapsulation yield and subsequent release efficiency.

The concentration of the pectin solution is advantageously from 4 to 10% (w/v), preferably 4 to 7%, if zinc acetate is selected, then the zinc solution is advantageously from 2 to 20% (w/v), preferably from 5 to 15%. More preferably, the pectin solution is about 5% (w/v), the zinc acetate solution is about 12% (w/v).

The pectin beads are advantageously stirred in the zinc salt solution at a pH that is most suited to optimize stability of active agent (usually around 5 to 7.5), at room temperature under slow agitation for at least 12 minutes up to 20 hours, preferably from 20 minutes to 2 hours.

The beads can then be recollected and rinsed in distilled water until conductivity of the rinsing solution reaches a plateau. Rinsing is preferably done at least twice or under a continuous process to minimize the amount of residual zinc salt recovered in the rinsing solution.

The rinsed beads can then be collected and subjected to a drying process using methods known to those of skill in the art, including heated incubator or fluid bed technologies.

The beads are preferably dried at a temperature of between 20 and 40° C. for 30 min to 24 hours, preferably at 35° C. overnight. Drying is performed preferentially until the weight of the beads reaches a plateau.

The diameter of the particles can be finely tuned using needles of appropriate internal diameter to form the pectin drops added to the zinc salt solution. The beads are preferably between about 600 and 1500 μm in diameter.

The encapsulation yields are usually close to 100%, depending on the aqueous solubility of the active agent to be encapsulated.

### IV. Formation of Drug Delivery Systems Including Pectin Beads

The pectin beads can be collected, and combined with appropriate excipients and formulated into a variety of oral drug delivery systems. For example, the beads can be combined with a solid excipient, and tableted, or included in a capsule.

The pectin beads can also be combined with liquid/gel excipients which do not degrade the pectin beads, and the mixture/dispersion can be incorporated into a capsule, such as a gel-cap.

The tablets or capsules can be coated, if desired, with a suitable enteric coating so as to assist in passing through the stomach without degradation. The pH in the stomach is of the order of 1 to 3 but it increases in the small intestine and the colon to attain values close to 7 (Hovgaard L. et al. (1996) Current Applications of Polysaccharides in Colon Targeting, Critical Reviews in Therapeutic Drug Carrier Systems, 13, 185). The drug delivery systems, in the form of tablets, gelatin capsules, spheroids and the like, can reach the colon, without being exposed to these variations in pH, by coating them with a pH-dependent polymer, insoluble in acidic pH but soluble in neutral or alkaline pH (Kinget et al., op. cit.). The polymers most currently used for this purpose are derivatives of methacrylic acid, Eudragit® L and S (Ashford M. et al. (1993), An in vivo investigation of the suitability of pH-dependent polymers for colonic targeting, International Journal of Pharmaceutics, 95, 193 and 95, 241; and David A. et al. (1997) Acrylic polymers for colon-specific drug delivery, S.T.P. Pharma Sciences, 7, 546).

The drug delivery systems are administered in an effective amount suitable to provide the adequate degree of treatment or prevention of the disorders for which the compounds are administered. The efficient amounts of these compounds are typically below the threshold concentration required to elicit any appreciable side effects. The compounds can be administered in a therapeutic window in which some the disorders are treated and certain side effects are avoided. Ideally, the effective dose of the compounds described herein is sufficient to provide the desired effects in the colon but is insufficient (i.e., is not at a high enough level) to provide undesirable side effects elsewhere in the body.

Most preferably, effective doses are at very low concentrations, where maximal effects are observed to occur, with minimal side effects, and this is optimized by targeted colonic delivery of the active agents. The foregoing effective doses typically represent that amount administered as a single dose, or as one or more doses administered over a 24-hour period.

The toxicity and therapeutic efficacy of the active agents described herein can be determined by standard pharmaceutical procedures in cell cultures or experimental animals, e.g., by determining the LD₅₀, (the dose lethal to 50% of the population) and the ED₅₀ (the dose therapeutically effective in 50% of the population). The dose ratio between toxic and therapeutic effect is the therapeutic index and can be expressed as the ratio between LD₅₀ and ED₅₀. Compounds which exhibit high therapeutic indices are preferred. The data obtained from these cell culture assays and animal studies can be used in formulating a dosage range that is not toxic for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p. 1).

Dosage amount and interval may be adjusted individually to provide levels of the active compounds in the colon which are sufficient to maintain therapeutic effect. Preferably, therapeutically effective levels will be achieved by administering multiple doses each day. One of skill in the art will be able to optimize therapeutically effective local dosages without undue experimentation.

### V. Methods of Treatment Using the Drug Delivery Systems Described Herein

The drug delivery systems described herein can be used to treat colonic infections, and disorders such as diarrhea which result from exposure of the colon to bacterial toxins.

The active agents can be administered in a therapeutically effective dosage to a patient who has been, or is likely to suffer from, a colonic infection.

The compositions and methods can be used to treat a variety of colonic bacterial infections, or colonization by potentially pathogenic and/or multi-resistant bacteria or yeasts, as well as various disorders resulting from bacterial infections (i.e., those caused by toxins produced by such bacterial infections such as bacterial or parasitic dysenteria or colitis. The microorganisms which can be treated using the drug delivery systems described herein include Gram-positive bacteria such as Staphylococcus aureus, vancomycin resistant enterococci, Clostridium difficile, and Clostridium botulinum, Gram-negative bacteria such as Escherichia coli and other species of enterobacteria particularly those multiresistant to antibiotics, Salmonella and shigella species and as well as other potentially pathogenic strict aerobes bacteria such as Pseudomonas aeruginosa, Stenotrophomanas maltophilia, Acinetobacter baumannii and the like, and fungi such as Candida albicans and other species of Candida.

Microbial growth can be inhibited by administering an effective amount of a suitable active agent in a properly designed delivery system and dosage form (for example, bacteriophage, phage protein, antibody, antibody fragment, antimicrobial peptide and/or aptamer) to a patient in need of such treatment. The exact dose to be administered will vary according to the use of the compositions, age, sex and physiologic condition of the patient, and can readily be determined by the treating physician. The compositions may be administered as a single dose or in a continuous manner over a period of time. Doses may be repeated as appropriate.

The compositions can be used to provide antimicrobial agents to the colon to eliminate pathogenic microbe within the lumen of the intestinal tract, minimizing the pathogenic alterations of the mucosa resulting from the action of compounds (such as toxins) released by infecting bacteria such as Clostridium difficile. Thus, the compositions can be used to treat post-antibiotic diarrhea, colitis, or pseudo-membranous colitis, which are commonly grouped under the name of Clostridium difficile associated diseases, or CDAD.

The compositions can also be used to provide antimicrobial agents to the colon to provide “selective decontamination,” eliminating commensal and/or potentially pathogenic microorganisms (such as for example enterobacteria, pseudomonas, enterococci) from the colon of patients at risk (such as Intensive care, or haemato-oncology patients, for example) before they develop an actual infection.

The compositions can further be used to provide selective decontamination of the colonic bacteria in farm animals, particularly, of specific types of Escherichia coli strains, namely Shiga-toxin Escherichia coli (or STEC), also called Verotoxin Escherichia coli (or VETEC). In use, the practice of this method can minimize contamination of the food and water supplies.

The compositions can also be used to provide selective decontamination against colonization by vancomycin resistant enterococci (VRE), providing selective decontamination using anti-VRE molecules specifically targeted to the colon.

The compositions can also be used to provide selective decontamination to help control outbreaks of antibiotic-resistant gram-negative infections, such as nosocomial infections, in hospitals. Representative infections include nosocomial infections caused by enterobacteria (mostly Klebsiella) resistant to third generation cephalosporins by secretion of an extended spectrum beta-lactamase (ESBL) derived from the TEM or SHV beta-lactamase families, as well as a new type of ESBL, called CTX-M, which have a strikingly different epidemiological pattern of emergence and diffusion. In one embodiment, patients admitted to a hospital, who have identified positive for one of these bacteria, are selectively decontaminated using specific antibacterial agents targeted to the colon.

Ideally, the antimicrobial active agent(s) delivered to the colon are specific for pathogenic bacteria, and do not have an effect on beneficial bacteria. In this manner, the agents do not cause selective pressure in the colon, and the beneficial bacteria are preserved. Accordingly, in one embodiment, the proteins, peptides, or aptamers are active against one or more species of harmful bacteria present in the colon, but are either not active, or are less active, against helpful bacteria present in the colon. It can be particularly useful to use more than one agent, so that the problem of bacterial resistance developing against one agent is minimized.

Although it is desirable to selectively treat pathogenic bacteria, rather than beneficial bacteria, in one embodiment, the active agents are non-selective, and eliminate both beneficial and pathogenic bacteria, and the beneficial bacteria can be replaced, for example, using probiotic formulations.

The following table summarizes examples of the types of treatments, and the types of active agents most suited for each type of treatment.

| | Active agent | | | |
|---|---|---|---|---|
| | Classical | Peptide | bacterio- | |
| Target | antibiotic | antibiotic | phage | antibodies |
| Treatment of | no | Yes | yes | Yes |
| C difficile | | | | against |
| infection | | | | C. difficile |
| and CDAD | | | | toxin |
| Selective | Colymycin | Yes | No | no |
| decontam- | Aminoglycosides | | (too many | |
| ination | | | different | |
| against Gram | | | targets) | |
| negative rods | | | | |
| Decontam- | no | Yes | yes | no |
| ination | | | | |
| of livestock | | | | |
| of STEC | | | | |
| Decontam- | no | Yes | No | no |
| ination | | | (to many | |
| of vancomycin | | | different | |
| resistant | | | targets) | |
| enterococci | | | | |

| | |
|---|---|
| Control of | Same as for selective decontamination |
| CTX-M | against Gram negative rods |

The present invention will be better understood with reference to the following non-limiting examples.

### Example 1

### Composition for Colonic Delivery of an Antimicrobial Peptide

A large number of peptides have been described that exert antimicrobial activity on Gram positive bacteria. Many such peptides are from natural origin, and some have been modified to optimize their antimicrobial activity. A given peptide may be specific for a particular bacterial species (or even strain or group of strains), or exert activity against a larger group of microorganisms.

The purpose of this invention is to target such peptides to the colon so that they can locally eliminate potentially dangerous colonizing bacteria such as enterocoques (and in particular strains resistant to glycopeptides, usually known as vancomycin-resistant enterococci or VRE), Clostridium difficile and Staphylococcus aureus.

A dosage form that would enable the oral administration of such peptides, protect them during gastro-intestinal transit from acidic pH and proteolytic cleavage, and release them intact at the level of the distal ileum or caecum (proximal colon) would enable to deliver directly to the site of infection high concentrations of the active antimicrobial peptide.

Purified peptide (produced by any method, including purified from natural or genetically modified sources, synthetic, or any combination thereof) is mixed with a 5% solution of pectin (amidated or non amidated pectin). The solution is adjusted in terms of pH and ionic strength to avoid any degradation of the active agent and is added dropwise to a solution of calcium or zinc chloride (counterion) at a concentration so that pectin gelifies into beads of 2-5 mm diameter. After gelification, beads are rinsed in purified water for to remove excess counterion. Beads are then dried between 25 and 35° C. to prevent any active agent degradation during drying and to remove any residual water. Beads can be screened according to size and further coated by a first layer of neutral polymer such as hydroxypropyl methyl cellulose (HPMC) or carboxymethyl cellulose (CMC) to smooth the bead surface and allow for a better grip of the enteric coating. This coating could represent 5 to 20% of final beads' weight. A final coat is made of a gastro-resistant, entero-soluble polymer such as Eudragit L30D-55, Eudragit FS30D or a combination with NE30D polymers to achieve sustained released or controlled release properties (usual ratios ranging from 2:1 to 1:2). The final coat represents between 20% and 40% of the beads' final weight. In particular, the solution is added dropwise to a solution of ZnCl2 at 7.45% (0.55 M), so that pectin gelifies into beads of 2-3 mm diameter. After gelification, beads are rinsed twice in purified water for 20 min until conductivity remains at a constant value. Beads are then dried at 35° C. for up to 19 h, and are then separated according to size; those beads in the interval 0.8-1 mm are selected for further use. Beads are then successively coated by a first layer of hydroxypropyl methyl cellulose (HPMC) representing 5% of the beads' weight, and then by a mixture of Eudragit L30D-55 and NE30D polymers (in ratios ranging from 3:7 to 3:8) representing between 25% and 36% of the beads' final weight. Eudragit solutions may contain such additives as Tween 80 (usually around 0.3%), glycerol monostearate (usually around 0.8%), and triethyl citrate (usually around 0.8%). HPMC and Eudragit solutions are sprayed onto the beads at 33° C. in a fluid bed apparatus and further dried in the air flow at the same temperature. Beads are packaged in capsules for oral administration.

Eudragit solutions may contain such additives as Tween 80 (usually around 0.3%), glycerol monostearate (usually around 0.8%), and triethyl citrate (usually around 0.8%). HPMC and Eudragit solutions are sprayed onto the beads at 33° C. in a fluid bed apparatus and further dried in the air flow at the same temperature. Beads are packaged in capsules for oral administration.

### Example 2

### Composition for Colonic Delivery of an Isolated Bacteriophage Lysin

Double stranded DNA bacteriophages lyse their host bacteria cells by digesting the major structural polymer of the bacterial cell wall, peptidoglycan, with specialized enzymes encoded by the bacteriophage's genome, lysins. Such isolated enzymes can be used to lyse Gram positive bacteria with great efficacy. Some lysins are highly specific of a given bacterial specied (or even strain), whereas others are active on a broader group of bacteria.

The purpose of this invention is to target such lysins to the colon so that they can locally eliminate potentially dangerous colonizing bacteria such as enterocoques (and in particular strains resistant to glycopeptides, usually known as vancomycin-resistant enterococci or VRE), Clostridium difficile and Staphylococcus aureus. A dosage form that would enable the oral administration of such proteins, protect them during gastro-intestinal transit from acidic pH and proteolytic cleavage, and release them intact at the level of the distal ileum or caecum (proximal colon) would enable to deliver directly to the site of infection high concentrations of the active bacteriophage proteins.

Purified lysin (produced by any method, including purified from natural or recombinant bacteria) is mixed with a 5% solution of pectin (amidated or non amidated pectin). The solution is adjusted in terms of pH and ionic strength to avoid any degradation of the active agent and is added dropwise to a solution of calcium or zinc chloride (counterion) at a concentration so that pectin gelifies into beads of 2-5 mm diameter. After gelification, beads are rinsed in purified water for to remove excess counterion. Beads are then dried between 25 and 35° C. to prevent any active agent degradation during drying and to remove any residual water. Beads can be screened according to size and further coated by a first layer of neutral polymer such as hydroxypropyl methyl cellulose (HPMC) or carboxymethyl cellulose (CMC) to smooth the bead surface and allow for a better grip of the enteric coating. This coating could represent 5 to 20% of final beads' weight. A final coat is made of a gastro-resistant, entero-soluble polymer such as Eudragit L30D-55, Eudragit FS30D or a combination with NE30D polymers to achieve sustained released or controlled release properties (usual ratios ranging from 2:1 to 1:2). Final coat is representing between 20% and 40% of the beads' final weight.

Eudragit solutions may contain such additives as Tween 80 (usually around 0.3%), glycerol monostearate (usually around 0.8%), and triethyl citrate (usually around 0.8%). HPMC and Eudragit solutions are sprayed onto the beads at 33° C. in a fluid bed apparatus and further dried in the air flow at the same temperature. Beads are packaged in capsules for oral administration.

### Example 3

### Composition for Colonic Delivery of Antibodies Targeted Against Bacterial Toxins

Clostridium difficile is a Gram positive spore-forming bacillus that is the leading cause of nosocomial antibiotic-associated diarrhea because of the disruption of the colonic flora due to antibiotic treatments. C difficile-associated diarrhea (CDAD) is mediated by two exotoxins produced by the bacteria, toxin A and toxin B. Both are large proteins (280 to 310 kDa) that possess multiple functional domains: an N-terminal enzymatic domain that carries a glucosyltransferase activity that modifies low molecular weight GTPases leading to the cytotoxic effects of the toxins, a central domain thought to be involved in membrane transport, and a C-terminal domain believed to interact with carbohydrate receptors present at the surface of target cells. Both toxins are cytotoxic, though toxin B is 1,000-fold more active than toxin A in in vitro toxicity assays, and both are lethal when injected intravenously of intraperitoneally into mice.

Neutralizing these toxins would constitute a potent method to alleviate the symptoms of CDAD. One such method would be to vectorize to the colon neutralizing antibodies that would inhibit the cytotoxic activity of toxin A and toxin B. Such antibodies have indeed been produced, and a recent study has shown that human monoclonal antibodies, injected intraperitoneally into hamsters, were efficient in reducing C difficile-induced mortality (Babcock, et al, Infection and Immunity, 2006, 74, 6339-6347). A dosage form that would enable the oral administration antibodies capable of neutralizing toxins A and B from C difficile, protect them during gastro-intestinal transit from acidic pH and proteolytic cleavage, and release them intact at the level of the distal ileum or caecum (proximal colon) would enable to deliver directly to the site of infection high concentrations of the active antibodies to neutralize the harmful bacterial toxins.

Purified antibodies or antibody fragments (produced by any method, including purified from hybridomas or recombinant bacteria) is mixed with a 5% solution of pectin (amidated or non amidated pectin). The solution is adjusted in terms of pH and ionic strength to avoid any degradation of the active agent and is added dropwise to a solution of calcium or zinc chloride (counterion) at a concentration so that pectin gelifies into beads of 2-5 mm diameter. After gelification, beads are rinsed in purified water for to remove excess counterion. Beads are then dried between 25 and 35° C. to prevent any active agent degradation during drying and to remove any residual water. Beads can be screened according to size and further coated by a first layer of neutral polymer such as hydroxypropyl methyl cellulose (HPMC) or carboxymethyl cellulose (CMC) to smooth the bead surface and allow for a better grip of the enteric coating. This coating could represent 5 to 20% of final beads' weight. A final coat is made of a gastro-resistant, entero-soluble polymer such as Eudragit L30D-55, Eudragit FS30D or a combination with NE30D polymers to achieve sustained released or controlled release properties (usual ratios ranging from 2:1 to 1:2). Final coat is representing between 20% and 40% of the beads' final weight.

Eudragit solutions may contain such additives as Tween 80 (usually around 0.3%), glycerol monostearate (usually around 0.8%), and triethyl citrate (usually around 0.8%). HPMC and Eudragit solutions are sprayed onto the beads at 33° C. in a fluid bed apparatus and further dried in the air flow at the same temperature. Beads are packaged in capsules for oral administration.

### Example 4

### Composition for Colonic Delivery of an Antibiotic

There are circumstances in which it would be highly desirable to selectively remove unwanted bacteria such as C difficile, enterococci, Pseudomonas, or certain strains of E coli producing toxins (such as Shiga toxin or Verotoxin) from the colon of humans or animals. In this example, one would choose to deliver an antibiotic, or a mixture of antibiotics, directly to the colon.

Colistin (polymyxin E) is a polymyxin antibiotic produced by certain strains of Bacillus polymyxa var. colistinus. Colistin is a mixture of cyclic polypeptides colistin A and B. Colistin is effective against Gram-negative bacilli, except Proteus and Burkholderia cepacia, and is used as a polypeptide antibiotic.

Polypeptide antibiotics are a class of antibiotics that are normally considered toxic, and are therefore not suitable for systemic administration, and are typically administered topically on the skin or by inhalation. Examples include actinomycin, bacitracin, colistin, and polymyxin B. As used herein, specific delivery to the colon overcomes the systemic toxicity problems, and enables the compounds to treat colonic infections as described herein.

Polypeptide antibiotic formulation can be prepared by mixing the drug to an excipient for dry or wet granulation such as cellulose microcrystalline (Avicel PH 101 or 102 for example) and a disintegrant such as Crospovidone to improve the release characteristics after compression.

Such a granulation mixture is then dried and further crushed into a powder that can be compressed into tablets or extruded into pellets. Tablets or pellets are then coated with a final coat consisting of a gastro-resistant, entero-soluble polymer such as Eudragit L30D-55, Eudragit FS30D or a combination with NE30D polymers to achieve sustained released or controlled release properties. Final coat is representing between 5% and 20% of tablets' final weight.

Eudragit solutions may contain such additives as Tween 80 (usually around 0.3%), glycerol monostearate (usually around 0.8%), and triethyl citrate (usually around 0.8%).

### Example 5

### Effect of Zinc Concentration and Drying Time on the Stability of Beads in Simulated Intestinal Media (SIM)

Beads containing an active agent can be prepared as previously described, and gelled with 6 or 12% zinc salt solutions. The effect of drying time can be tested by drying beads for 2, 4 and 16 h at 35° C. (the preferred temperature is 37° C. for industrialization purposes). The release of active agents from the resulting beads can then be evaluated.

### Example 6

### Effect of Gelification Time, Rinsing Process, and Drying Time on Recovery of Active Agents

Different batches of beads can be prepared using a multi-nozzle system from Nisco Engineering AG. The beads can undergo various gelification times, rinsing processes and times, and drying process types and times. The release of active agents from the resulting beads can then be evaluated.

### Example 7

### Composition for Colonic Delivery of an Antimicrobial Peptide

A large number of peptides have been described that exert antimicrobial activity on Gram positive bacteria. Many such peptides are from natural origin, and some have been modified to optimize their antimicrobial activity. A given peptide may be specific for a particular bacterial species (or even strain or group of strains), or exert activity against a larger group of microorganisms.

The purpose of this invention is to vectorize such peptides to the colon so that they can locally eliminate potentially dangerous colonizing bacteria such as enterocoques (and in particular strains resistant to glycopeptides, usually known as vancomycin-resistant enterococci or VRE), Clostridium difficile and Staphylococcus aureus. A dosage form that would enable the oral administration of such peptides, protect them during gastro-intestinal transit from acidic pH and proteolytic cleavage, and release them intact at the level of the distal ileum or caecum (proximal colon) would enable to deliver directly to the site of infection high concentrations of the active antimicrobial peptide.

Purified peptide (produced by any method, including purified from natural or genetically modified sources, synthetic, or any combination thereof) is mixed with a 5% solution of pectin (amidated or non amidated pectin). The solution is adjusted in terms of pH and ionic strength to avoid any degradation of the active agent and is added dropwise to a solution of calcium or zinc chloride (counterion) at a concentration so that pectin gelifies into beads of 2-5 mm diameter. After gelification, beads are rinsed in purified water for to remove excess counterion. Beads are then dried between 25 and 35° C. to prevent any active agent degradation during drying and to remove any residual water. Beads can be screened according to size and further coated by a first layer of neutral polymer such as hydroxypropyl methyl cellulose (HPMC) or carboxymethyl cellulose (CMC) to smooth the bead surface and allow for a better grip of the enteric coating. This coating could represent 5 to 20% of final beads' weight. A final coat is made of a gastro-resistant, entero-soluble polymer such as Eudragit L30D-55, Eudragit FS30D or a combination with NE30D polymers to achieve sustained released or controlled release properties (usual ratios ranging from 2:1 to 1:2). Final coat is representing between 20% and 40% of the beads' final weight.

Eudragit solutions may contain such additives as Tween 80 (usually around 0.3%), glycerol monostearate (usually around 0.8%), and triethyl citrate (usually around 0.8%). HPMC and Eudragit solutions are sprayed onto the beads at 33° C. in a fluid bed apparatus and further dried in the air flow at the same temperature. Beads are packaged in capsules for oral administration.

### Example 8

### Composition for Colonic Delivery of an Isolated Bacteriophage Lysin

Double stranded DNA bacteriophages lyse their host bacteria cells by digesting the major structural polymer of the bacterial cell wall, peptidoglycan, with specialized enzymes encoded by the bacteriophage's genome, lysins. Such isolated enzymes can be used to lyse Gram positive bacteria with great efficacy. Some lysins are highly specific of a given bacterial specied (or even strain), whereas others are active on a broader group of bacteria.

The purpose of this invention is to vectorize such lysins to the colon so that they can locally eliminate potentially dangerous colonizing bacteria such as enterocoques (and in particular strains resistant to glycopeptides, usually known as vancomycin-resistant enterococci or VRE), Clostridium difficile and Staphylococcus aureus. A dosage form that would enable the oral administration of such proteins, protect them during gastro-intestinal transit from acidic pH and proteolytic cleavage, and release them intact at the level of the distal ileum or caecum (proximal colon) would enable to deliver directly to the site of infection high concentrations of the active bacteriophage proteins.

Purified lysin (produced by any method, including purified from natural or recombinant bacteria) is mixed with a 5% solution of pectin (amidated or non amidated pectin). The solution is adjusted in terms of pH and ionic strength to avoid any degradation of the active agent and is added dropwise to a solution of calcium or zinc chloride (counterion) at a concentration so that pectin gelifies into beads of 2-5 mm diameter. After gelification, beads are rinsed in purified water for to remove excess counterion. Beads are then dried between 25 and 35° C. to prevent any active agent degradation during drying and to remove any residual water. Beads can be screened according to size and further coated by a first layer of neutral polymer such as hydroxypropyl methyl cellulose (HPMC) or carboxymethyl cellulose (CMC) to smooth the bead surface and allow for a better grip of the enteric coating. This coating could represent 5 to 20% of final beads' weight. A final coat is made of a gastro-resistant, entero-soluble polymer such as Eudragit L30D-55, Eudragit FS30D or a combination with NE30D polymers to achieve sustained released or controlled release properties (usual ratios ranging from 2:1 to 1:2). Final coat is representing between 20% and 40% of the beads' final weight.

Eudragit solutions may contain such additives as Tween 80 (usually around 0.3%), glycerol monostearate (usually around 0.8%), and triethyl citrate (usually around 0.8%). HPMC and Eudragit solutions are sprayed onto the beads at 33° C. in a fluid bed apparatus and further dried in the air flow at the same temperature. Beads are packaged in capsules for oral administration.

### Example 9

### Composition for Colonic Delivery of Antibodies Targeted Against Bacterial Toxins

Clostridium difficile is a Gram positive spore-forming bacillus that is the leading cause of nosocomial antibiotic-associated diarrhea because of the disruption of the colonic flora due to antibiotic treatments. C difficile associated diarrhea (CDAD) is mediated by two exotoxins produced by the bacteria, toxin A and toxin B. Both are large proteins (280 to 310 kDa) that possess multiple functional domains: an N-terminal enzymatic domain that carries a glucosyltransferase activity that modifies low molecular weight GTPases leading to the cytotoxic effects of the toxins, a central domain thought to be involved in membrane transport, and a C-terminal domain believed to interact with carbohydrate receptors present at the surface of target cells. Both toxins are cytotoxic, though toxin B is 1,000-fold more active than toxin A in in vitro toxicity assays, and both are lethal when injected intravenously of intraperitoneally into mice.

Neutralizing these toxins would constitute a potent method to alleviate the symptoms of CDAD. One such method would be to vectorize to the colon neutralising antibodies that would inhibit the cytotoxic activity of toxin A and toxin B. Such antibodies have indeed been produced, and a recent study has shown that human monoclonal antibodies, injected intraperitoneally into hamsters, were efficient in reducing C difficile-induced mortality (Babcock, et al, Infection and Immunity, 2006, 74, 6339-6347). A dosage form that would enable the oral administration antibodies capable of neutralising toxins A and B from C difficile, protect them during gastro-intestinal transit from acidic pH and proteolytic cleavage, and release them intact at the level of the distal ileum or caecum (proximal colon) would enable to deliver directly to the site of infection high concentrations of the active antibodies to neutralise the harmful bacterial toxins.

Purified antibodies or antibody fragments (produced by any method, including purified from hybridomas or recombinant bacteria) are mixed with a 5% solution of pectin (amidated or non amidated pectin). The solution is adjusted in terms of pH and ionic strength to avoid any degradation of the active agent and is added dropwise to a solution of calcium or zinc chloride (counterion) at a concentration so that pectin gelifies into beads of 2-5 mm diameter. After gelification, beads are rinsed in purified water for to remove excess counterion. Beads are then dried between 25 and 35° C. to prevent any active agent degradation during drying and to remove any residual water. Beads can be screened according to size and further coated by a first layer of neutral polymer such as hydroxypropyl methyl cellulose (HPMC) or carboxymethyl cellulose (CMC) to smooth the bead surface and allow for a better grip of the enteric coating. This coating could represent 5 to 20% of final beads' weight. A final coat is made of a gastro-resistant, entero-soluble polymer such as Eudragit L30D-55, Eudragit FS30D or a combination with NE30D polymers to achieve sustained released or controlled release properties (usual ratios ranging from 2:1 to 1:2). Final coat is representing between 20% and 40% of the beads' final weight.

Eudragit solutions may contain such additives as Tween 80 (usually around 0.3%), glycerol monostearate (usually around 0.8%), and triethyl citrate (usually around 0.8%). HPMC and Eudragit solutions are sprayed onto the beads at 33° C. in a fluid bed apparatus and further dried in the air flow at the same temperature. Beads are packaged in capsules for oral administration.

### Example 10

### Composition for Colonic Delivery of an Antibiotic

There are circumstances in which it would be highly desirable to selectively remove unwanted bacteria such as C difficile, enterococci, Pseudomonas, or certain strains of E coli producing toxins (such as Shiga toxin or Verotoxin) from the colon of humans or animals. In this example, one would choose to deliver an antibiotic, or a mixture of antibiotics, directly to the colon.

All documents cited above are hereby incorporated in their entirety by reference. From the foregoing, it will be obvious to those skilled in the art that various modifications in these methods and compositions can be made without departing from the spirit and scope of the invention. Accordingly, the invention may be embodied in other specific forms without departing from the spirit or essential characteristics thereof. Present embodiments and examples, therefore, are to be considered in all respects as illustrative and not restrictive, and all changes which come within the meaning and range of equivalency of the claims are therefore intended to be embraced therein. All documents referred to herein are hereby incorporated by reference.

1. A composition for treating bacterial infection, eliminating unwanted bacterial colonization in the colon, and/or removing bacterial toxins, comprising a drug delivery system that is orally administered, and delivers specifically to the colon, and substantially avoids delivery to areas other than the colon, wherein the drug delivery system comprises one or more active agents selected from the group consisting of:
a) a polypeptide antibiotic,
b) an aminoglycoside, and
c) an agent which binds to or inactivates a bacterial toxin.

2. (canceled)

3. The composition of claim 2, wherein the polypeptide antibiotic is colistin.

4. The composition of claim 1, wherein the polypeptide antibiotic or aminoglycoside is selective for harmful (pathogenic) bacteria over helpful (beneficial) bacteria.

5. The composition of claim 1, wherein the active agent is an antibody which binds to bacterial toxins.

6. The composition of claim 1, wherein the drug delivery system comprises two or more active agents.

7. The composition of claim 1, wherein the drug delivery system comprises pectin beads that encapsulate the active agent(s).

8. The composition of claim 1, wherein the active agent is capable of lysing the bacterial cell wall.

9-11. (canceled)

12. The composition of claim 1, wherein the system comprises beads of pectin in the form of a cationic salt enclosing the active agent.

13. The composition of claim 6, wherein the pectin beads are further coated by a cationic or a pH dependent polymer such as Eudragit® type polymers.

14. A composition for treating bacterial infection, eliminating unwanted bacterial colonization in the colon, and/or removing bacterial toxins, comprising a drug delivery system that is orally administered, and delivers specifically to the distal ileum, wherein the drug delivery system comprises one or more active agents selected from the group consisting of:
a) a polypeptide antibiotic,
b) an aminoglycoside, and
c) an agent which binds to or inactivates a bacterial toxin.

15. The composition of claim 14, wherein the active agent is selective for harmful (pathogenic) bacteria over helpful (beneficial) bacteria.

16. The composition of claim 14, wherein the active agent is an agent which binds to bacterial toxins.

17. The composition of claim 14, wherein the drug delivery system comprises two or more active agents.

18. The composition of claim 14, wherein the drug delivery system comprises pectin beads that encapsulate the active agent(s).

19. The composition of claim 14, wherein the active agent is capable of lysing the bacterial cell wall.

20-22. (canceled)

23. The composition of claim 14, wherein the system comprises beads of pectin in the form of a cationic salt enclosing the active agent.

24. The composition of claim 23, wherein the pectin beads are further coated by a cationic or a pH dependent polymer such as Eudragit® type polymers.

25. A method of eliminating pathogenic microbes within the lumen of the intestinal tract, and minimizing the pathogenic alterations of the mucosa resulting from the action of compounds (such as toxins) released by infecting bacteria, comprising administering to a patient in need of treatment thereof a composition of claim 1.

26. A method of treating Clostridium difficile infection, or Clostridium difficile associated diseases (CDAD), comprising administering to a patient in need of treatment thereof a composition of claim 1.

27. A method for providing selective decontamination to the colon of a patient at risk (such as intensive care, or haemato-oncology patients) before they develop an actual infection, comprising administering to a patient in need of treatment thereof a composition of claim 1.

28. A method for providing selective decontamination of the colonic bacteria in farm animals, comprising administering to a farm animal a composition of claim 1.

29. The method of claim 28, wherein the colonic bacteria to be selectively decontaminated are Shiga-toxin Escherichia coli (or STEC).

30. A method for providing selective decontamination against colonization by vancomycin resistant enterococci (VRE), comprising administering to a patient in need of treatment thereof a composition of claim 1, wherein the active agent is an anti-VRE molecule.

31. A method for providing selective decontamination to control outbreaks of antibiotic-resistant gram-negative infections, such as nosocomial infections, in hospitals, comprising administering to a patient in need of treatment thereof a composition of claim 1.

32. The method of claim 31, wherein the nosocomial infection is caused by a) CTX-M, or b) enterobacteria which is resistant to third generation cephalosporins by secretion of an extended spectrum beta-lactamase (ESBL) derived from the TEM or SHV beta-lactamase families.

33. The method of claim 31, wherein patients admitted to a hospital, who have identified positive for one of these bacteria, are selectively decontaminated using specific antibacterial agents targeted to the colon.

34. The method of claim 25, wherein the active agents are active against one or more species of harmful bacteria present in the colon, but are either not active, or are less active, against helpful bacteria present in the colon.

35. The method of claim 25, wherein more than one agent is used, so that the problem of bacterial resistance developing against one agent is minimized.

36. The method of claim 25, wherein the active agents are non-selective, and eliminate both beneficial and pathogenic bacteria.

37. The method of claim 36, further comprising replacing the beneficial bacteria by using a probiotic formulation.

38. A method of reducing the concentration of bacteria in the colon of a patient, comprising orally administering the drug delivery system of claim 1 to a patient who has a colonic bacterial infection, or who is at risk of having a colonic bacterial infection.

39-42. (canceled)

43. The method of claim 38, wherein the system comprises beads of pectin in the form of a cationic salt enclosing the active agent.

44. The method of claim 43, wherein the pectin beads are further coated by a cationic or a pH dependent polymer such as Eudragit® type polymers.

45-50. (canceled)
